(19) United States
(12) Patent Application Publication
(10) Pub. No.: US 2003/0013643 A1
(43) Pub. Date: Jan. 16, 2003

(54) Prevention and treatment of streptococcal and staphylococcal infection

(76) Inventors: Michael R. Wessels, Brookline, MA (US); Colette Cywes, Brookline, MA (US)

Correspondence Address: Edward R. Gates, c/o Wolf, Greenfield & Sacks, P.C., Federal Reserve Plaza, 600 Atlantic Avenue, Boston, MA, 02210-2211, US

(21) Appl. No.: 10/005,200

(22) Filed: Dec. 5, 2001

Related U.S. Application Data
(63) Continuation-in-part of application No. 09/960,921, filed on Sep. 25, 2001, now Pat. No. 6,467,419.
(60) Provisional application No. 60/234,145, filed on Sep. 21, 2000.

(51) Int. Cl. A61K 38/16; A61K 31/728

(52) U.S. Cl. 514/8; 514/54

(57) ABSTRACT

The invention provides new methods for use in prevention and treatment of streptococcal and staphylococcal infection.

### RELATED APPLICATIONS

This application is a continuation-in-part of U.S. application Ser. No. 09/960,621, filed Sept. 21, 2001, now pending, and claims the benefit under 35 U.S.C. §119(e) from U.S. provisional application serial No. 60/234,145, filed Sept. 21, 2000.

### GOVERNMENT SUPPORT

This invention was made in part with government support under grant number A129952 from the National Institutes of Health (NIH). The government may have certain rights in this invention.

### FIELD OF THE INVENTION

The invention relates to the prevention and treatment of streptococcal and staphylococcal infection.

### BACKGROUND IF THE INVENTION

Group A streptococcus (also known as Streptococcus pyogenes or GAS) and Group C streptococcus (GCS) are common human pathogens. Group A streptococcus is believed to colonize the pharynx leading directly to clinical manifestations such as streptococcal pharyngitis (strep throat), and indirectly to infections at other anatomic sites such as cellulitis or necrotizing fasciitis (hemolytic streptococcal gangrene); or to systemic infections such as streptococcal toxic shock syndrome, scarlet fever, sepsis, and bacteremia. Indirectly, GAS may also lead to post-infectious syndromes of acute rheumatic fever or glomerulonephritis. Group C streptococci, which may also colonize the pharynx, may result in human infections similar to those caused by Group A streptococci, including pharyngitis, pneumonia, cellulitis and soft-tissue infection, bacteremia, septic arthritis, and endocarditis. Group C bacteria may play a role in the aforementioned types of infections in the elderly or chronically ill patients; and in addition to its role as a human pathogen, Group C streptococcus is also a major pathogen in animals such as horses.

The onset of a disease, such as one of the aforementioned illnesses, is a complex event that results from factors including bacterial colonization, invasion, toxin elaboration, and the host's responses to these factors. One element in colonization is bacterial adhesion to a mucosal surface (Harrison's Principles of Internal Medicine, 1998). The pharynx, which is a mucosal surface, is believed to be a “reservoir” for GAS and GCS colonization and concomitant disease. In addition, to symptomatic infection, streptococcal infections are commonly asymptomatic, which means that although not clinically ill, the subject has pharyngeal colonization and is a carrier of the infection and capable of spreading the infection to others. Pharyngeal colonization may result in secondary infections and disease, as described herein. For example, even in the absence of clinical symptoms, bacteria asymptomatically colonizing the pharynx may be transferred from the throat to other regions of the body, resulting in infection. Therefore, treating GAS and GCS infections may comprise treating symptomatic disease and/or may comprise treating asymptomatic colonization.

CD44 has been identified as a receptor for Group A streptococcus. A monoclonal antibody to CD44 (IM7.8.1) has been shown in vitro to prevent binding, also known as adhesion, of Group A streptococcal bacteria (five types) to the receptor. Streptococcal bacteria are believed to attach to CD44 via the bacterial capsule, which is composed of hyaluronic acid. Although these findings serve to elucidate possible mechanisms of streptococcal binding in vitro, these results do not serve as a model for in vivo binding because significant differences between in vitro and in vivo environments exist. For example, the in vivo pharyngeal tissue, unlike the tissue in culture, is coated with a mucous layer, which makes hyaluronic acid access to CD44 on the pharyngeal cells unpredictable. In addition, presence of a wide assortment of cell and receptor types in the in vivo pharyngeal environment presents numerous obstacles to hyaluronic acid binding in vivo that are unparalleled in the simple binding parameters at work in the in vitro studies. These types of differences between the in vitro and in vivo environments render the findings from in vitro experiments not predictive of functional interference with disease and not suggestive of viable methods for in vivo applications. In short, the in vitro assays are not accepted models for in vivo binding behavior and efficacy.

Hyaluronic acid in the capsule is a simple linear polymer of β(1→4)-linked repeating units of β-D-glucuronic acid(1→3) β-D-N-acetylglucosamine, and is structurally identical to mammalian hyaluronic acid. Hyaluronic acid has been utilized extensively in the cosmetic and pharmaceutical industry for numerous applications such as: lubrication of prosthetics, replacement fluids in eye surgery, an anti-inflammatory in arthritic joints, an anti-cancer agent, to prevent cavities, and as a carrier to allow other pharmaceuticals to access desirable sites of action in the body, but its administration, according to the invention, to reduce the likelihood of streptococcal infections, such as GAS and GCS, and staphylococcal infections is novel and unexpected.

Hyaluronic acid has been utilized in a number of pharmaceutical-type products. For example, hyaluronic acid has also been hypothesized to work synergistically with Echinacea to stop upper respiratory infections and is included as an ingredient in a formulation called Thronase.® The manufacturer of Thronase® suggests that Thronase® relies on the activity of Echinacea to reduce infection, and that this activity is enhanced by the inclusion of hyaluronic acid in the formulation. The prior art relating to Thronase® teaches administrating Echinacea in combination with hyaluronic acid to achieve a synergistic effect, and teaches away from the use of hyaluronic acid alone. (In contrast, in the methods and products of the invention described herein, hyaluronic acid is administered to a subject as an active ingredient, and surprisingly is effective as a prophylactic agent or as a treatment to inhibit colonization of the pharynx and subsequent infection by bacteria.). The dose hyaluronic acid administered in conjunction with Echinacea as taught in the prior art is a total dose of hyaluronic acid of 0.16 mg, administered over a two hour time period.

### SUMMARY OF THE INVENTION

The invention involves the surprising discovery that agents that bind to the hyaluronic acid binding region of CD44 can interfere in vivo with adhesion, colonization, and disease caused by streptococcal bacteria. The invention also involves the discovery of the criticality of does of hyaluronic acid in preventing adhesion, colonization and disease.

The invention provides methods and products related to the treatment of streptococcal and staphylococcal infections.

According to one aspect of the invention, there is provided a method for treating a subject to reduce the likelihood of streptococcal infection. The method involves administering orally to a subject in need of such treatment an agent that binds to a hyaluronic acid-binding region of a CD44 protein an amount effective to interfere with adhesion of streptococcal bacteria to CD44 protein in the subject and inhibit streptococcal colonization of the pharynx, wherein either one or both of the following conditions applies: the treatment is free of Echinacea or the agent is administered in a dose greater than 0.2 mg. In one embodiment, the effective amount of agent administered statistically reduces the likelihood of infection. The agent can be administered to a subject suspected of exposure to streptococcal bacteria, likely to be exposed to streptococcal bacteria, or known to have been exposed to streptococcal bacteria. In particular, embodiments of streptococcal bacteria are Group A streptococcus or Group C streptococcus. Preferably, the subject is a human. In some embodiments, the subject is in need of treatment to reduce the likelihood of streptococcal pharyngitis. In certain embodiments, the CD44 protein in the subject in need of such treatment is located on the pharynx.

The agent can be any agent that binds to a hyaluronic acid-binding region of a CD44 protein. In some embodiments, the agent is a peptide. In other embodiments, the agent is an antibody. In yet other embodiments the agent is hyaluronic acid or an analog of hyaluronic acid. Preferably, the agent is hyaluronic acid.

The dose can be a single oral administration of hyaluronic acid or multiple oral administrations of hyaluronic acid. In certain embodiments, the dose is at least 0.2 mg administered in under 2 hours. In other embodiments, the dose is at least 0.2 mg administered in under 1 hour, under 30 minutes, and even under 15 minutes. In certain embodiments, the dose is at least 0.25 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 10.0 mg hyaluronic acid.

In certain embodiments, the doses administered in under 1.5 hours, 1.0 hours, 45 minutes, 30 minutes, 10 minutes, 5 minutes, 4 minutes, 3 minutes, 2 minutes, 1 minute, 30 seconds, 15 seconds, 10 seconds, 5 seconds, or 1 second.

In any of the foregoing embodiments, the treatment can be free of Echinacea.

According to another aspect of the invention, a method is provided for treating a subject to reduce the likelihood of streptococcal infection. The method involves administering orally to a subject administering orally to a subject in need of such treatment an agent that binds to a hyaluronic acid-binding region of a CD44 protein in an amount effective to displace streptococcal bacteria to CD44 protein in the subject and inhibit streptococcal colonization of the pharynx, wherein either one or both of the following conditions applies: the treatment is free of Echinacea or the agent is administered in a dose greater than 0.2 mg. The various embodiments, including effective amounts, preferred subjects, agents, dosages, and times of administration are as discussed above.

According to yet another embodiment, a method is provided for treating a subject to reduce the likelihood of streptococcal or staphylococcal infection. The method involves administering orally to a subject in need of such treatment an agent that binds to a hyaluronic acid-binding region of a CD44 protein of a mucosal membrane in an amount effective to interfere with adhesion of streptococcal or staphylococcal bacteria to the mucosal membrane in the subject, wherein either one or both of the following conditions applies: the treatment is free of Echinacea or the agent is administered in a dose greater than 0.2 mg. In one embodiment, the hyaluronic acid interferes with adhesion of streptococcal bacteria to CD44 protein of a mucosal membrane in the subject. In one embodiment, the effective amount of agent administered statistically reduces the likelihood of infection. The agent can be administered to a subject suspected of exposure to Group A streptococcal, Group C streptococcal, Group B streptococcal, streptococcus pneumoniae, or staphlycoccal aureus bacteria, likely to be exposed to Group A streptococcal, Group C streptococcal, Group B streptococcal, streptococcus pneumoniae, or staphylococcal aureus bacteria, or known to have been exposed to Group A streptococcal, Group C streptococcal, Group B streptococcal, streptococcus pneumoniae, or staphlycoccal aureus bacteria. In particular, embodiments of streptococcal bacteria are Group A streptococcus or Group C streptococcus. Preferably, the subject is a human. In some embodiments, the subject is in need of treatment to reduce the likelihood of streptococcal or staphylococcal pharyngitis. In certain embodiments, the CD44 protein in the subject in need of such treatment is located on the pharynx.

The agent can be any agent that binds to a hyaluronic acid-binding region of a CD44 protein. In some embodiments, the agent is a peptide. In other embodiments, the agent is an antibody. In yet other embodiments the agent is hyaluronic acid or an analog of hyaluronic acid. Preferably, the agent is hyaluronic acid.

The dose can be a single oral administration of hyaluronic acid or multiple oral administrations of hyaluronic acid. In certain embodiments, the dose is at least 0.2 mg administered in under 2 hours. In other embodiments, the dose is at least 0.2 mg administered in under 1 hour, under 30 minutes, and even under 15 minutes. In certain embodiments, the dose is at least 0.25 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 10.0 mg hyaluronic acid.

In certain embodiments, the doses administered in under 1.5 hours, 1.0 hours, 45 minutes, 30 minutes, 10 minutes, 5 minutes, 4 minutes, 3 minutes, 2 minutes, 1 minute, 30 seconds, 15 seconds, 10 seconds, 5 seconds, or 1 second.

In any of the foregoing embodiments, the treatment can be free of Echinacea.

According to yet another aspect of the invention, a method is provided for treating a subject to reduce the likelihood of streptococcal or staphylococcal infection. The method involves administering orally to a subject in need of such treatment an agent that binds to a hyaluronic acid-binding region of a CD44 protein of a mucosal membrane in an amount effective to displace streptococcal or staphylococcal bacteria bound to the mucosal membrane in the subject, wherein either one or both of the following conditions applies: the treatment is free of Echinacea or the agent is administered in a dose greater than 0.2 mg. The various embodiments, including effective amounts, preferred subjects, agents, dosages, and times of administration are as discussed in connection with the immediately-preceding aspect of the invention.

According to another aspect of the invention, a method is provided to reduce the likelihood of streptococcal or staphylococcal infection. The method involves administering nasally to a subject in need of such treatment who does not have a nasal cavity wound, an agent that binds to a hyaluronic acid-binding region of a CD44 protein of a mucosal membrane in an amount effective to interfere with adhesion of streptococcal or staphylococcal bacteria to the mucosal membrane in the subject, wherein either one or both of the following conditions applies: the treatment is free of Echinacea or the agent is administered in a dose greater than 0.2 mg. The various embodiments, including effective amounts, preferred subjects, agents, dosages, and times of administration are as discussed in connection with the immediately-preceding aspect of the invention.

According to another aspect of the invention, a method is provided for treating a subject to reduce the likelihood of streptococcal or staphylococcal infection. The method involves administering nasally to a subject in need of such treatment who does not have a nasal cavity wound, an agent that binds to a hyaluronic acid-binding region of a CD44 protein of a mucosal membrane in an amount effective to displace streptococcal or staphylococcal bacteria bound to the mucosal membrane in the subject, wherein either one or both of the following conditions applies: the treatment is free of Echinacea or the agent is administered in a dose greater than 0.2 mg.

The various embodiments, including effective amounts, preferred subjects, agents, dosages, and times of administration are as discussed in connection with the immediately-preceding aspect of the invention.

According to another aspect of the invention, there is provided a medicinal product. The product is a syrup containing an amount of hyaluronic acid effective to interfere with adhesion of streptococcal bacteria to CD44 protein in a subject and to inhibit streptococcal colonization of a pharynx of the subject.

Another aspect of the invention is a medicinal product that is a syrup containing an amount of hyaluronic acid effective to displace streptococcal bacteria bound to CD44 protein in a subject and to inhibit streptococcal colonization of a pharynx of the subject.

Another aspect of the invention is a medicinal product that is a syrup containing an amount of hyaluronic acid effective to interfere with adhesion of streptococcal or staphylococcal bacteria to a mucosal membrane in a subject. This mucosal membrane can be a pharynx of the subject.

Another aspect of the invention is a medicinal product that is a syrup containing an amount of hyaluronic acid effective to displace streptococcal or staphylococcal bacteria bound to mucosal membrane in a subject. The mucosal membrane can be a pharynx of the subject.

Another aspect of the invention is a medicinal product that is frozen solution containing an amount of hyaluronic acid effective to interfere with adhesion of streptococcal or staphylococcal bacteria to CD44 In protein in a subject and to inhibit streptococcal colonization of a pharynx of the subject.

Another aspect of the invention is a medicinal product that is a frozen solution containing an amount of hyaluronic acid effective to displace streptococcal bacteria bound to CD44 protein in a subject and to inhibit streptococcal colonization of a pharynx in the subject.

Another aspect of the invention is a medicinal product that is frozen solution containing an amount of hyaluronic acid effective to interfere with adhesion of streptococcal or staphylococcal bacteria to a mucosal membrane in a subject. The mucosal membrane in the subject can be a pharynx of the subject.

Another aspect of the invention is a medicinal product that is a frozen solution containing an amount of hyaluronic acid effective to displace streptococcal or staphylococcal bacteria bound to a mucosal membrane in the subject. The mucosal membrane in the subject can be a pharynx of the subject.

Another aspect of the invention is a medicinal product that is a solid solution containing an amount of hyaluronic acid effective to interfere with adhesion of streptococcal bacteria to CD44 protein in a subject and to inhibit streptococcal colonization of a pharynx of the subject.

Another aspect of the invention is a medicinal product that is a solid solution containing an amount of hyaluronic acid effective to displace streptococcal bacteria bound to a CD44 protein in a subject and to inhibit streptococcal colonization of a pharynx of the subject.

Another aspect of the invention is a medicinal product that is a solid solution containing an amount of hyaluronic acid effective to interfere with adhesion of streptococcal or staphylococcal bacteria to a mucosal membrane in a subject The mucosal membrane in the subject can be a pharynx of the subject.

Another aspect of the invention is a medicinal product that is a solid solution containing an amount of hyaluronic acid effective to displace streptococcal or staphylococcal bacteria bound to a mucosal membrane in a subject. The mucosal membrane in the subject can be a pharynx of the subject.

Another aspect of the invention is a medicinal product that is a semi-solid solution containing an amount of hyaluronic acid effective to interfere with adhesion of streptococcal bacteria to CD44 protein in a subject and to inhibit streptococcal colonization of a pharynx of the subject.

Another aspect of the invention is a medicinal product that is a semi-solid solution containing an amount of hyaluronic acid effective to displace streptococcal bacteria bound to CD44 protein in a subject and to inhibit streptococcal colonization of a pharynx of the subject.

Another aspect of the invention is a medicinal product that is a semi-solid solution containing an amount of hyaluronic acid effective to interfere with adhesion of streptococcal or staphylococcal bacteria to a mucosal membrane in a subject. The mucosal membrane in the subject can be a pharynx of the subject.

Another aspect of the invention is a medicinal product that is a semi-solid solution containing an amount of hyaluronic acid effective to displace streptococcal or staphylococcal bacteria bound to a mucosal membrane in a subject. The mucosal membrane in the subject can be a pharynx of the subject.

In any of the foregoing embodiments, the medicinal product can be fashioned as a single dose which contains at least wherein the dose is at least 0.20 mg, 0.25 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 10.0 mg hyaluronic acid.

The solid solution medicinal product, semi-solid medicinal produce, syrup medicinal product, and the frozen solution medicinal product are described in greater detail below in the Detailed Description.

These and other aspects of the invention will be described in greater detail below in connection with the drawings.

### BRIEF DESCRIPTION OF THE DRAWINGS

FIG. 1 is a graph illustrating the inhibition of GAS attachment to normal mouse keratinocytes by mAb to CD44. Data represent mean ±SD of adherent bacteria recovered after inoculation of keratinocytes with GAS in the absence (filled bars) or presence (hatched bars of anti-CD44 mAb KM81, 15 μg/ml. KM81-treatment inhibited attachment of wild-type (encapsulated) GAS strains B514-Sm and 950771, but not the acapsular mutant strain 188.

FIG. 2 is a graph demonstrating GAS attachment to wild-type or CD44-deficient mouse keratinocytes. Data represent mean ±SD of adherent bacteria recovered after inoculation of wild-type (solid bars) or K5-CD44 antisense (CD44-deficient, hatched bars) keratinocytes with wild-type (encapsulated) GAS strain B514-Sm or 950771 or with their respective acapsular mutants, UAB039 and 188. Attachment of the wild-type GAS strains, but not the acapsular mutant strains, to CD44-deficient keratinocytes was reduced by approximately 75% compared to attachment to wild-type keratinocytes.

FIG. 3 illustrates GAS pharyngeal colonization of wild-type mice and of mice deficient in expression of epithelial CD44. (A) is a photomicrographic image of histologic section through the pharynx of a representative wild-type mouse stained with mAb to CD44. Immunohistochemical staining of the pharyngeal epithelium is seen with mAb to CD44, but not with an irrelevant control mAb. Labels indicate the location of the epithelium (E), lumen (L), and submucosa (S). (B) is a table in which the left column shows the level of CD44 expression in the pharyngeal epithelium of wild-type and K5-CD44 antisense mice. Histologic sections were scored for CD44 expression in the pharyngeal epithelium by 3 independent observers without knowledge of the throat culture results. CD44 expression was graded from 1 (background) to 4 (equivalent to wild-type control). On the right are results of throat cultures for GAS on each of 5 days after intranasal inoculation with GAS B514-Sm. (C) is a histogram of summary of throat culture results presented in panel B. Data represent the percentage of mice with a positive throat culture on each day after intranasal inoculation for wild-type mice (solid bars), transgenic mice with wild-type levels of CD44 on keratinocytes (hatched bars), and transgenic mice with reduced or absent CD44 expression on keratinocytes (open bars).

FIG. 4 is two graphs that illustrate the prevention of GAS pharyngeal colonization in vivo by anti-CD44 monoclonal antibody. Data represent the fraction of mice with positive throat cultures for GAS on each of 3 days after intranasal inoculation with GAS administered either with mAb to CD44 or with an irrelevant control mAb (n=20 mice per group).

FIG. 5 is three graphs that demonstrate inhibition of GAS attachment to mouse keratinocytes by exogenous hyaluronic acid. Data represent mean ±SD of adherent bacteria recovered after incubation of keratinocytes with GAS in the presence of no inhibitor, hyaluronic acid (HA), or a control polysaccharide, alginic acid (AL), at the indicated times after addition of GAS to the keratinocytes.

FIG. 6 is a bar graph that illustrates the effective concentration of hyaluronic acid to inhibit bacterial binding and the effective concentration of hyaluronic acid to displace bacterial binding.

FIG. 7 is a graph showing the effect of hyaluronic acid added exogenously in preventing adhesion of pathogenic bacteria to human keratinocytes.

FIG. 8 is three graphs that illustrate the prevention of GAS pharyngeal colonization by pretreatment with hyaluronic acid. Data represent the fraction of mice with positive throat cultures for GAS on each of 3 days after intranasal inoculation with GAS following pretreatment with phosphate-buffered saline, hyaluronic acid (HA), or a control polysaccharide, alginic acid (AL) (n=13 mice per group).

FIG. 9 is a bar graph that shows the result of pretreatment of keratinocytes with hyaluronic acid and alginic acid as the percent blocking of translocation of Group A streptococcus at 1 and 2 hours after inoculation.

FIG. 10 is a bar graph showing the effect of pretreatment of abraded skin with 2 mg/ml hyaluronic acid or 2 mg/ml alginic acid for one hour to assess the percent inhibition of translocation of Group A streptococcus.

FIG. 11 is a bar graph that illustrates the effects of Group A Streptococcal bacteria in human keratinocytes.

FIG. 12 shows three bar graphs illustrating translocation of GAS through polarized keratinocyte monolayers. (A) the mean number of colony-forming units recovered from the medium beneath the monolayer at indicated times after inoculation of the apical surface with wild-type (solid bars) or acapsular (hatched bars) GAS. *P=0.0002 for comparison with the wild-type strain. (B) Internalization of GAS by keratinocytes in a polarized monolayer. At indicated time points after inoculation of the apical surface of the monolayer, extracellular GAS were killed by addition of penicillin and gentamicin, and intracellular GAS were recovered after lysis of the keratinocytes. Data represent mean colony-forming units of intracellular GAS recovered from monolayers inoculated with wild-type (solid bars) or acapsular (hatched bars) GAS. *P=0.0002 for comparison with acapsular strain. (C) Translocation of GAS through human skin equivalent. Data represent mean number of colony-forming units of GAS recovered from beneath a sample of human skin equivalent (see text) at various times after inoculation of the epidermal surface with wild-type (solid bars) or acapsular (hatched bars) GAS. *P=0.008 for comparison with the wild-type strain.

FIG. 13 is four drawings of which (A) is a syrup medicinal product (8) with syrup (12) in a container (10), (B) is a solid solution medicinal product (14), with a solid solution (16), and a handle (18), (C) is a frozen solution medicinal product (20), with a frozen solution (22), and a handle (24), and (D) is a semi-solid solution medicinal product (30) with a semi-solid solution (32).

### DETAILED DESCRIPTION OF THE INVENTION

The invention is based on the surprising finding that the oral administration of hyaluronic acid results in hyaluronic acid binding to CD44 and blocking the adhesion and colonization of streptococcal and staphylococcal bacteria such as Group A streptococcus, Group B streptococcus, Group C streptococcus, streptococcus pneumoniae, and staphylococcus aureus in the pharynx and nasal mucosa. Accordingly, the invention is related to new uses for hyaluronic acid in subjects, in view of its newly discovered function, namely a role in in vivo reduction in streptococcal and staphylococcal binding and colonization in the pharynx, a known reservoir for streptococcal infection. The invention also relates to the use of other agents that bind to the hyaluronic acid binding region of CD44, such as nonpeptide organic agents and peptides, for in vivo reduction of streptococcal and staphylococcal adhesion and colonization of the pharynx and to the use of the agents to reduce the likelihood of Group A streptococcus, Group B streptococcus, Group C streptococcus, streptococcus pneumoniae, and staphylococcus aureus binding and infection.

As used herein, a subject is a human, non-human primate, cow, horse, pig, sheep, goat, dog, cat, or rodent. In all embodiments human subjects are preferred. Particularly important subjects to which the present invention can be applied are subjects diagnosed as having been or suspected of having been exposed to Group A streptococcus, Group B streptococcus, Group C streptococcus, streptococcus pneumoniae, and staphylococcus aureus, which includes subjects diagnosed with infection, exhibiting symptoms of infection, or having known or probable risk of exposure to an individual known to have streptococcal or staphylococcal infection. For example, close family members of an individual testing positive for streptococcal or staphylococcal infection would be suspected of having been exposed to or at risk of future exposure to streptococcal or staphylococcal bacteria.

A subject may or may not exhibit symptoms of infection such as streptococcal pharyngitis, which is also known as strep throat. If present, symptoms may include sore throat, fever, chills, vomiting, swelling of the pharyngeal mucosa, and purulent exudate over the posterior pharyngeal wall and tonsillar pillars (Harrison's Principles of Internal Medicine, 1998). Methods to diagnose symptomatic and asymptomatic streptococcal or staphylococcal infection are known to those of ordinary skill in the medical arts and include, but are not limited to, swab of affected region for bacterial culture or rapid streptococcal diagnostic testing such as the latex agglutination or enzyme immunoassay of swab specimens. Infected regions may include the throat, nasal mucosa, or other body regions. In an important embodiment of the invention the infection is in the pharynx.

Treatment as it relates to the invention may be prophylactic or post-infection. Prophylactic treatment may comprise administering hyaluronic acid, a hyaluronic acid analog, or other agent that binds the hyaluronic acid binding region of CD44, to interfere with streptococcal binding, which is also known as adhesion, to CD44 protein in the subject. Post-exposure or post-infection treatment may comprise administering hyaluronic acid, a hyaluronic acid analog, or other agent that binds to the hyaluronic acid binding region of CD44 to displace streptococcal adhesion to CD44 protein in the subject. Thus, in an important embodiment, the hyaluronic acid, analog, or other agent that binds to the hyaluronic acid binding region of CD44 is administered to inhibit colonization (i.e. further binding) or even to displace streptococcal binding and/or to interfere with streptococcal binding in a subject. As used herein, the term “inhibit colonization” means to reduce or lower the level of colonization. To inhibit may also mean to prevent colonization, but it is not necessary to prevent all colonization to lessen or prevent the manifestation of disease.

Agents can be prepared that bind specifically to CD44 and/or the hyaluronic acid binding region of CD44. As used herein, “binding specifically to the hyaluronic acid binding region of CD44” means binding to and distinguishing the hyaluronic acid binding region of CD44 from other regions of CD44. As used herein, “binding specifically to CD44” means binding to and distinguishing CD44 from other naturally occurring proteins. Agents that bind to CD44 molecules, including but not limited to the hyaluronic acid binding region of CD44 molecules, include hyaluronic acid, also known as sodium hyaluronate, analogs thereof, and organic agents. Polypeptide agents include polyclonal and monoclonal antibodies, prepared according to conventional methodology. Certain antibodies useful in the methods of the invention already are known in the art and include certain anti-CD44 antibodies, for example the monoclonal antibody IM7.8. 1, which binds to CD44.

An “agent that binds to the hyaluronic acid binding region of CD44” is one that binds to the hyaluronic acid-binding site on CD44 and interferes with streptococcal binding to the site, or binds close enough to the hyaluronic binding site to interfere with streptococcal binding. Such agents include nonpeptide and peptide agents. Such agents can be organic agents identified, for example, using routine combinatorial screening methodology or through immunological procedures. Since the binding site and its ligand are known, rationally based combinatorial libraries can be prepared, based on computer-generated models of the binding site and/or 3-dimensional structural information obtained from crystals of the bound complex.

Such agents also can be synthesized chemically from peptides or other biomolecules including but not limited to saccharides, fatty acids, sterols, isoprenoids, purines, pyrimidines, derivatives or structural analogs of the above, or combinations thereof and the like. Phage display libraries and chemical combinatorial libraries can be used to develop and select synthetic compounds which bind to the hyaluronic acid binding region of CD44. Also envisioned in the invention is the use of agents made from peptoids, random bio-oligomers (U.S. Pat. No. 5,650,489), benzodiazepines, diversomeres such as dydantoins, benzodiazepines and dipeptides, nonpeptidal peptidomimetics with a beta-D-glucose scaffolding, oligocarbamates or peptidyl phosphonates.

The agents of the invention thus may be produced en masse using library technology. In some aspects, the methods of the invention utilize this library technology to generate and subsequently identify small molecules, including small peptides, that bind to the hyaluronic acid binding region of CD44 molecules. One advantage of using libraries is the facile manipulation of millions of different putative candidates of small size in small reaction volumes (i.e., in synthesis and screening reactions). Another advantage of libraries is the ability to synthesize agents which might not otherwise be attainable using naturally occurring sources, particularly in the case of non-peptide moieties. A “molecular library” refers to a collection of structurally-diverse molecules. Molecular libraries can be chemically-synthesized or recombinantly produced. As used herein, a “molecular library member” refers to a molecule that is present within the molecular library. In general, a molecular library contains from two to 10¹² molecules, and any integer number therebetween, e.g., 2, 3, 4, 5, 10, 10², 10³, 10⁴, 10⁵, 10⁶, 10⁷, 10⁸, 10⁹, 10¹⁰10¹¹, 10¹² and so forth, as if each and every integer has been recited herein.

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties which are less subject to enzymatic degradation compared to their naturally-occurring counterparts. Libraries are also meant to include for example but are not limited to peptide or plasmid libraries, polysome libraries, aptamer libraries, synthetic peptide libraries, synthetic small molecule libraries and chemical libraries. The libraries can also comprise cyclic carbon or heterocyclic structure and/or aromatic or polyaromatic structures substituted with one or more of the above-identified functional groups.

Many, if not all, of these agents of the invention can be synthesized using recombinant or chemical library approaches. A vast array of candidate agonists or antagonists can be generated from libraries of synthetic or natural compounds. Libraries of natural compounds in the form of bacterial, fungal, plant and animal extracts are available or can readily produced. Natural and synthetically produced libraries and compounds can be readily modified through conventional chemical, physical, and biochemical means. Known binding partners of CD44 molecules may be subjected to directed or random chemical modifications such as acylation, alkylation, esterification, amidification, etc. to produce structural analogs of these binding partners. Synthetic DNA and RNA libraries are also commonly used in the art. For instance, Ellington and Szostak describe the use of random polynucleotide libraries to identify novel ligands [Ellington and Szostak, Nature, 346, 818-822 (1990)].

As described in U.S. Pat. No. 5,908,609, exemplary library compounds also include, but are not limited to, peptides such as, for example, soluble peptides, including but not limited to members of random peptide libraries; (see, e.g., Lam, K. S. et al. 1991, Nature 354:82-84;Houghten, R. et al. 1991, Nature 354:84-86), and combinatorial chemistry-derived molecular libraries made of D- and/or L-configuration amino acids, phosphopeptides (including but not limited to, members of random or partially degenerate, directed phosphopeptide libraries; (see, e.g., Songyang, Z. et al. 1993, Cell 72: 767-778), antibodies (including, but not limited to, polyclonal, monoclonal, humanized, anti-idiotypic, chimeric or single chain antibodies, and FAb, F(ab′)₂ and FAb expression library fragments (and epitope-binding fragments thereof), and small organic or inorganic molecules. Compounds that can be designed to satisfy the foregoing criteria include polypeptides and peptide mimetics. The peptide mimetic can be a hybrid molecule which includes both amino acid and non-amino acid components, e.g., the mimic can include amino acid components for the positively charged and negatively charged regions and a non-amino acid (e.g., piperidine) having the same approximate size and dimension of a hydrophobic amino acid (e.g., phenylalanine) as the hydrophobic component.

Small molecule combinatorial libraries may also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application W095/18972, published Jul. 13, 1995 and U.S. Pat. No. 5,712,171 granted Jan. 27, 1998 and its corresponding PCT published patent application W096/22529, which are hereby incorporated by reference.

Thus, according to still another aspect of the invention, low-molecular-weight compounds that inhibit the interaction between hyaluronic acid and a CD44 molecule are provided. These compounds can be used to modulate the interaction or can be used as lead compounds for the design of better compounds using the computer-based rational drug design methods.

Other methods for preparing or identifying peptides which bind to a particular target are known in the art. Molecular imprinting, for instance, may be used for the de novo construction of macromolecular structures such as peptides which bind to a particular molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Klaus Mosbach, Molecular Imprinting, Trends in Biochem. Sci., 19(9) January 1994; and Wulff, G., in Polymeric Reagents and Catalysts (Ford, W. T., Ed.) ACS Symposium Series No. 308, pp 186-230, American Chemical Society (1986). As an example, one method for preparing mimics of hyaluronic acid involves the steps of: (i) polymerization of functional monomers around a known substrate (the template or in this case, the hyaluronic acid- binding region) that exhibits a desired activity; (ii) removal of the template molecule; and then (iii) polymerization of a second class of monomers in the void left by the template, to provide a new molecule which exhibits one or more desired properties which are similar to that of the template. In addition to preparing peptides in this manner, other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are typically prepared by the free radical polymerization of functional monomers, resulting in a compound with a nonbiodegradable backbone. Other methods for designing such molecules include, for example, drug design based on structure activity relationships which require the synthesis and evaluation of a number of compounds and molecular modeling.

In yet another approach to the identification of hyaluronic acid mimics, antibodies which bind to the CD44 molecule are also embraced by the invention. Antibodies include polyclonal and monoclonal antibodies, prepared according to conventional methodology. Antibodies to the CD44 molecule can be generated using techniques standard in the art. Antibodies generated in such a manner can then be manipulated in a number of ways, as described below.

Another category relevant to interference with binding of hyaluronic acid and CD44 is peptides. Significantly, as is well-known in the art, only a small portion of an antibody molecule, the paratope, is involved in the binding of the antibody to its epitope (see, in general, Clark, W. R. (1986) The Experimental Foundations of Modem Inmmunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (Frs), which maintain the tertiary structure of the paratope (see, in general, Clark, W. R. (1986) The Experimental Foundations of Modem Immunology, Wiley & Sons, Inc., New York; Roift, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or Fr and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences. The present invention also includes so-called single chain antibodies.

Thus, the invention involves polypeptides of numerous size and type that bind specifically to the hyaluronic acid binding region of CD44 molecules and polypeptides that bind close enough to the hyaluronic acid bind region of CD44 to interfere with hyaluronic acid binding. These polypeptides may be derived also from sources other than antibody technology, as described above.

As mentioned above, peptides which bind to the hyaluronic acid or the CD44 molecule can be generated and identified by conventional screening methods such as phage display procedures (e.g., methods described in Hart, et al. J. Biol. Chem. 269:12468 (1994)). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to a receptor site are obtained by selecting those phages which express on their surface an amino acid sequence which recognizes and binds to the receptor. As used in this example, the CD44 molecule can be used as a receptor and the ligands are the peptides produced and displayed by the phage. These phage then are subjected to several cycles of reselection to identify those which have the most useful binding characteristics. The minimal linear portion of the sequence that binds to the ligand binding site can be determined. Typically, phage that exhibit the best binding characteristics (e.g., highest affinity) are further characterized by nucleic acid analysis to identify the particular amino acid sequences of the peptides expressed on the phage surface and the optimum length of the expressed peptide to achieve optimum binding.

One can repeat the procedure using a biased library containing inserts containing part of all of the minimal linear portion plus one or more additional degenerate residues upstream or downstream thereof. Yeast two-hybrid screening methods also may be used to identify polypeptides that bind to the CD44 molecules or hyaluronic acid binding region of CD44 molecules. Thus, CD44 molecules, or fragments thereof containing the hyaluronic acid binding region of CD44, can be used to screen peptide libraries, including phage display libraries, to identify and select peptide binding partners of the CD44 or hyaluronic acid binding region of CD44 molecules.

The displayed peptide sequence can vary in size. As the size increases, the complexity of the library increases. It is preferred that the total size of the displayed peptide sequence (the random amino acids plus any spacer amino acids) should not be greater than about 100 amino acids long, more preferably not greater than about 50 amino acids long, and most preferably not greater than about 25 amino acids long.

In certain embodiments, the libraries may have at least one constraint imposed upon the displayed peptide sequence. A constraint includes, but is not limited to, a positive or negative charge, hydrophobicity, hydrophilicity, a cleavable bond and the necessary residues surrounding that bond, and combinations thereof. In certain embodiments, more than one constraint is present in each of the peptide sequences of the library.

As mentioned above, the invention further embraces peptidomimetic residues, including non-naturally occurring amino acids. Such variants can be synthesized by substituting amino acids residues involved in the interaction between the peptide agent and the CD44 molecule with peptidomimetic residues. For example, glutamine (Glu) residues may be replaced with α-aminoadipate molecules and tyrosine positions may be substituted with 4-carboxymethyl-Phe. Phosphorus and non-phosphorus based analogs, such as phosphorotyrosine mimetics, may be used in the variants. Tyrosine analogs which can be used in place of the tyrosine residues include phenylalanine (Phe), pentafluoro phenylalanine (PfPhe), 4-carboxymethyl-L-phenylalanine (cmPhe), 4-carboxydifluoromethyl-L-phenylalanine (F₂cmPhe), 4-phosphonomethyl-phenylalanine (Pmp), (difluorophosphonomethyl)phenylalanine (F₂Pmp), O-malonyl-L-tyrosine (malTyr or OMT), and fluoro-O-malonyltyrosine (FOMT). Phosphonate-based mimetics which substitute a methylene unit for the tyrosyl phosphate ester bond may also be incorporated into synthetic agonists and antagonists. Additionally, glutamic acid residues can be modified to possess an additional methylene group or they may simply be substituted with α-amino-adipate (Adi). Other residues which may be used include the non-naturally occurring amino acid 1-aminocyclohexylcarboxylic acid (Ac₆c) and 3-(2-hydroxynaphtalen-1-yl)-propyl, or 2-azetidinecarboxylic acid or pipecolic acid (which have 6-membered, and 4-membered ring structures respectively) for proline residues, S-ethylisothiourea, 2-NH₂-thiazoline and 2-NH₂-thiazole. Also useful in the synthesis of variants is the use of asparagine residue substitutes such as 3-indolyl-propyl. It will be apparent to one of ordinary skill in the art that the invention embraces the synthesis of a wide variety of variants having any combination of amino acid analogs and/or peptidomimetic residues as described above and as are known in the art. Further potential modifications envisioned by the invention include modifications of cysteines, histidines, lysines, arginines, tyrosines, glutamines, asparagines, prolines, and carboxyl groups are well known in the art and are described in U.S. Pat. No. 6,037,134. Synthesis of the aforementioned variants is described in the cited references and is well within the realm of one of ordinary skill in the art.

The variants may also be modified to introduce or stabilize certain structural features. As an example, β-bends may be incorporated into the, preferably peptide, variants or the variants may be synthesized as cyclic peptides for example by incorporating thio-ether linkages.

The foregoing antibodies and other binding molecules may be used for example to isolate and identify CD44 protein and/or the hyaluronic acid binding region of CD44 protein. The antibodies may be coupled to specific diagnostic labeling agents for imaging of the protein or fragment thereof. The antibodies may also be used for immunoprecipitation, immunoblotting CD44 and/or hyaluronic acid binding region of CD44 molecules and/or in competitive binding assays using standard methods known to those of ordinary skill in the art.

Once a putative agent had been identified, it then can be tested in vitro in competitive binding assays with killed or live bacteria, and then in animals and humans, as described in the examples herein.

An “effective amount” of a treatment is that amount of hyaluronic acid, analog, or other agent that binds to the hyaluronic acid binding region of CD44 that alone, or together with further doses, produces the desired response, e.g. interference with Group A or Group C streptococcal binding to CD44 protein in the subject. In the case of treating a particular disease or condition, the desired response is inhibiting the onset or progression of the disease or condition, for example streptococcal pharyngitis. This may involve reducing but not necessarily preventing or eliminating the colonization of streptococcal or staphylococcal bacteria, although more preferably, it involves preventing or eliminating colonization of streptococcal or staphylococcal bacteria in the pharynx of the subject. The subject's response to treatment also may be a delay in the onset of the disease or condition.

The subject's response to treatment may be determined either directly or indirectly. Direct methods include, but are not limited to, standard swabbing and culturing, or rapid detection methods as described herein. Indirect methods of determining a subject's response may utilize statistical analysis based on the therapeutic responses to a plurality of dose and administration regimens administered to a plurality of subjects. For example, although not intended to be limiting is the comparison of dose and/or administration test values versus control values based for hyaluronic acid administration in a plurality of subjects. The responses of the test versus control groups may be compared and dose and/or administration regimen at which there is a statistically significant reduction in the likelihood of infection may be determined. Indirect methods may also include assessment of changes in symptoms of streptococcal or staphylococcal infection in a subject, as previously described herein. Other direct and indirect methods will be known to those of ordinary skill in the art and may be employed to assess the subject's response to treatment.

To elicit the desired response, treatment may be varied by increasing or decreasing the amount of a therapeutic composition, by changing the method of administration, by changing the dosage timing, by changing the pharmaceutical carrier, and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount may depend upon the degree to which a subject has colonization of streptococcal bacteria. For example, prophylactic treatment to prevent infection in an subjected suspected of exposure to streptococcal bacteria, in the presence of a positive or negative streptococcal culture, may comprise lower dosage and fewer administrations than treatment of a subject known to have streptococcal infection having a positive culture or test for streptococcal bacteria as described herein.

In one embodiment of the invention, an effective amount will be in the range from greater than 0.2 mg to 5000 mg hyaluronic acid, analog, or agent that binds the hyaluronic acid binding region of CD44. Preferably, an effective amount will be in the range from about 0.5 mg to 500 mg hyaluronic acid, analog, or agent that binds the hyaluronic acid binding region of CD44. Most preferably, an effective amount is about: 0.20 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, 10.0 mg, 11.0 mg, 12.0 mg, 13.0 mg, 14.0 mg, 15.0 mg, 20.0 mg, 25.0 mg, 30.0 mg, 35.0 mg, 40.0 mg, 50.0 mg, and all amounts up to 500 mg hyaluronic acid, analog, or agent that binds the hyaluronic acid binding region of CD44.

In some embodiments the effective amount of hyaluronic acid, analog or agent that binds the hyaluronic acid binding region of CD44 can be administered as a single dose. In other embodiments the dose is multiple administrations of hyaluronic acid. In other embodiments the effective amount of hyaluronic acid, analog or agent that binds the hyaluronic acid binding region of CD44 can be administered in more than one dose. An effective amount may be administered in doses administered over a period of time less than 2 hours, 1.5 hours, 1 hour, 45 minutes, 30 minutes, 15 minutes, 10 minutes, 5 minutes, 4 minutes, 3 minutes, 2 minutes, 1 minute, 30 seconds, 15 seconds, 10 seconds, 5 seconds or 1 second.

In some embodiments, doses of hyaluronic acid may be administered over an extended period for prophylactic purpose. An example of such a purpose, although not intended to be limiting is the administration of hyaluronic acid one or more times per day over a period of months, e.g. during the winter season, to prevent streptococcal infection.

In some embodiments, the average size of the hyaluronic acid is less than about 200 kDa, 150 kDa, 100 kDa, 50 kDa, 25 kDa, 20 kDa, 15 kDa, 10 kDa, or 5 kDa. In other embodiments, the average size of the hyaluronic acid is greater than about 200 kDa, 300 kDa, 400 kDa, 500 kDa, 600 kDa, 700 kDa, 800 kDa, 900 kDa, 1000 kDa, 5000 kDa, 10,000 kDA.

It is generally preferred that a maximum dose of the hyaluronic acid, analog, or other agent that binds to the hyaluronic acid binding region of CD44 (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of hyaluronic acid, analog, or other agent that binds to the hyaluronic acid binding region of CD44 administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Various modes of administration will be known to one of ordinary skill in the art which effectively deliver the hyaluronic acid, analog, or other agent that binds to the hyaluronic acid binding region of CD44 to a desired tissue, cell or bodily fluid. Administration may include but is not limited to: topical, intravenous, oral, intracavity, intrathecal, intrasynovial, buccal, sublingual, intranasal, transdermal, intravitreal, subcutaneous, intramuscular, and intradermal administration. Standard references in the art (e.g., Remington's Pharmaceutical Sciences, 18th edition, 1990) provide modes of administration and formulations for delivery of various pharmaceutical preparations and formulations in pharmaceutical carriers. The preferred method for administering the hyaluronic acid, analog, or other agent that binds to the hyaluronic acid binding region of CD44 is oral and most preferred is administration to the pharynx by swallowing. In other embodiments, the preferred method for administering the hyaluronic acid, analog, or other agent that binds to the hyaluronic acid binding region of CD44 is intranasal. Other protocols that are useful for the administration of hyaluronic acid, analog, or other agent that binds to the hyaluronic acid binding region of CD44 will be known to one of ordinary skill in the art, in which the dose amount, schedule of administration, sites of administration, mode of administration (e.g., intra-organ) and the like vary from those presented herein.

The pharmaceutical compositions used in the foregoing methods preferably are sterile, although this is not essential for oral or intranasal dosages, and contain an effective amount of hyaluronic acid, analog, or other agent that binds to the hyaluronic acid binding region of CD44 for producing the desired response in a unit of weight or volume suitable for administration to a patient.

Administration of hyaluronic acid, analog, or other agent that binds to the hyaluronic acid binding region of CD44 to mammals other than humans, e.g. for testing purposes or veterinary therapeutic purposes, is carried out under substantially the same conditions as described above. It will be understood by one of ordinary skill in the art that this invention is applicable to both human and animal diseases which can be treated by hyaluronic acid, analog, or other agent that binds to the hyaluronic acid binding region of CD44 . Thus this invention is intended to be used in husbandry and veterinary medicine as well as in human therapeutics.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Hyaluronic acid, analog, or other agent that binds to the hyaluronic acid binding region of CD44 may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the hyaluronic acid, analog, or other agent that binds to the hyaluronic acid binding region of CD44 , and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds.

The pharmaceutical compositions also may contain, optionally, suitable preservatives, such as: benzalkonium chloride; chlorobutanol; parabens, and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Preferably, pharmaceutical compositions are for oral or nasal administration. Pharmaceutical compositions for nasal administration may include: drops, suspensions, aerosols, inhalation sprays, and other nasal administration methods known to those of skill in the art. Compositions suitable for oral administration may be presented as discrete units, such as capsules, tablets, lozenges, chewing gum each containing a predetermined amount of the active compound. Preferred compositions include, but are not limited to: chewing gum, lozenges, and solutions or suspensions in aqueous liquids or non-aqueous liquids such as a syrup, elixir, or an emulsion. Preferred pharmaceutical compositions also include syrup such as a cough syrup, a solid solution (see U.S. Pat. No. 6,264,981), a frozen solution (see U.S. Pat. No. 6,258,384), or a semi-solid solution (see U.S. Pat. No. 5,932,273).

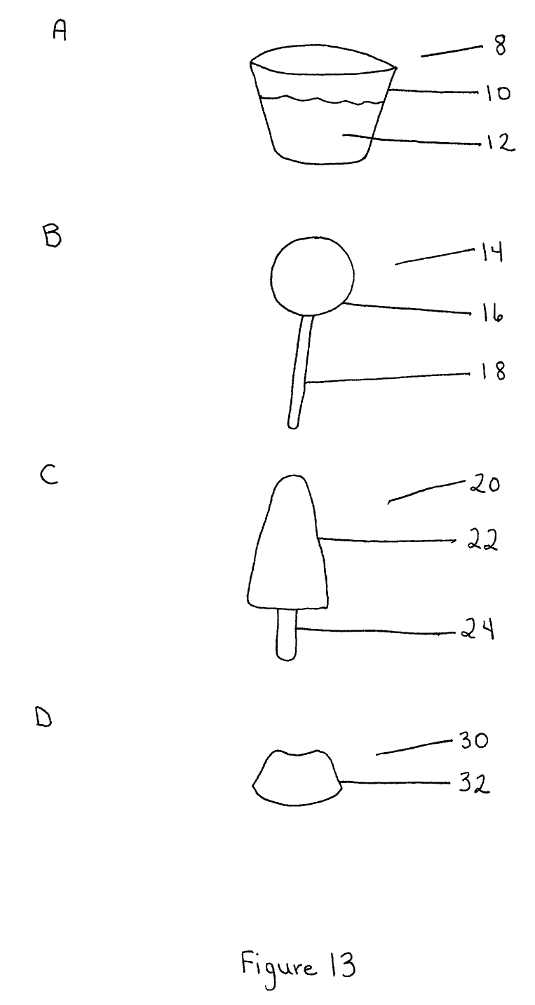

A syrup is thick liquid such as a mixture of sugar and water that is often flavored or medicated. An example of a syrup is cough syrup. A cough syrup is formulated typically with one or more of the following: dextromethorphan hydrobromide, pseudoephedrine hydrochloride, guaifenesin, acetaminophen, chlorpheniramine maleate, brompheniramine maleate, diphenhydramine hydrochloride, benzocaine, and dyclonine hydrochloride. Such ingredients may be distinguished from ingredients typically included in mouthwash and mouth rinse and toothpaste formulations, which include: thymol, eucalyptol, methyl salicylate, menthol, cetylpyridinium chloride and alcohol. Syrup ingredients may also be distinguished from ingredients typically included in mouth rinse and toothpaste formulations, which typically include sodium fluoride. A non-limiting example of a syrup medicinal product is given in FIG. 13A, in which 8 is the syrup medicinal product, 10 is the container, and 12 is the syrup.

A solid solution medicinal product is formulated typically with an aqueous base, dissolution agent, and pharmaceutical ingredient. A solid solution may also contain colorants, flavorants, solvents and co-solvents, coating agents, direct compression excipients, disintegrants, glidants, lubricants, opaquants, polishing agents, suspending agents, sweetening agents, anti-adherents, binders, and capsule diluents. The ingredients of a solid solution may also include preservatives, clarifying agents, emulsifying agents, antioxidants, levigating agents, plasticizers, surfactants, tonicity agents, and viscosity-increasing agents. The solid solution may be attached to a handle, an example of which, although not intended to be limiting, includes a stick or loop composed of wood, plastic, string, paper, metal, or other natural or synthetic material known to one of ordinary skill in the art. The solid solution may be on a platform that can be extended by the subject, for example, in the form of a push-up. It will be understood that the solid solution may be in any shape, and it may be of a size that can be placed in the mouth of the subject. In some embodiments the solid solution may be larger than can be placed in the mouth of the subject. A non-limiting example of a solid solution medicinal product is given in FIG. 13B, in which 14 is the solid solution medicinal product, 16 is the solid solution, and 18 is the handle.

A frozen solution medicinal product is formulated typically with active ingredients, flavoring agents, preservatives, food coloring, and a balance of water. The frozen solution medicinal product may be in a container, such as a cup or vessel, or a flexible sleeve, or it may be on a platform that can be extended by the subject, for example, in the form of a push-up. The frozen solution medicinal product may be attached to a handle, an example of which, although not intended to be limiting, includes a stick or loop composed of wood, plastic, string, paper, metal, or other natural or synthetic material known to one of ordinary skill in the art. The frozen solution medicinal product may be in any shape, and may be of a size that can be placed in the mouth of the subject. In some embodiments the frozen solution may be larger than can be placed in the mouth of the subject. A non-limiting example of a frozen solution medicinal product is given in FIG. 13C, in which 20 is the frozen solution medicinal product, 22 is the frozen solution, and 24 is the handle.

A semi-solid solution medicinal product is formulated typically with mixing active ingredients, water, sugar, starch syrup, an acid, a setting agent, a buffer solution, a flavoring material, and a coloring agent. Examples of setting agents, though not intended to be limiting are: pectin or gelatin. The semi-solid solution, also known as a gumi, gummi, or gummy, may be in any shape, and may be of a size that can be placed in the mouth of the subject. A non-limiting example of a semi-solid solution medicinal product is given in FIG. 13D, in which 30 is the semi-solid solution medicinal product and 32 is the semi-solid solution.

Compositions suitable for parenteral administration may be formulated according to known methods using suitable dispersing or wetting agents and suspending agents. The sterile injectable preparation also may be a sterile injectable solution or suspension in a nontoxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid may be used in the preparation of injectables. Carrier formulation suitable for oral, nasal, intrapulmonary, subcutaneous, intravenous, intramuscular, etc. administrations can be found in Remington 's Pharmaceutical Sciences, Mack Publishing Co., Easton, Pa., 18^{th}ed. 1990.

### EXAMPLES

### METHODS

### Examples 1-7

Bacterial Strains and Growth Conditions.

GAS strain B514-Sm is a spontaneous streptomycin-resistant derivative of B514/33, an M type 50 strain originally isolated from an epizootic infection of a mouse colony (Husmann, L. K., et al. 1996, Microb. Pathog. 20:213-24, Hook E. W., et al. 1960, Am. J. Hyg. 72:111-119), UAB039 is an acapsular mutant of B514-Sm constructed by insertion of a nonreplicating plasmid within the hasA (hyaluronate synthase) gene (Husmann, L. K. et al. 1997, Infect. Immun. 65: 1422-1430). GAS strain 950771 is an M type 3 strain originally isolated from a patient with necrotizing fasciitis; strain 188 is an acapsular mutant of 950771 constructed by insertion of the ΩKm2 element, a kanamycin-resistance cassette flanked by transcriptional terminators, within the hasA gene (Ashbaugh, C. D., et al. 1998, J. Clin. Invest. 102:550-560). Bacteria were grown in liquid culture in Todd-Hewitt broth or on trypticase soy agar containing 5% sheep blood. GAS were grown in liquid culture to mid-exponential phase (A₆₅₀ₙₘ=0.15), washed, and resuspended in serum-free keratinocyte basal medium without calcium or supplements (Clonetics, Inc., San Diego, Calif.) for in vitro attachment assays or in phosphate-buffered saline, pH 7.4, for intranasal inoculation of mice.

Mouse strains.

Unless otherwise specified, experiments involving mice were performed using C57BL/6 mice (Jackson Laboratories, Bar Harbor, Me.). K5-CD44 transgenic mice with a selective deficiency in expression of CD44 in stratified squamous epithelia have been described previously (Kaya, G. et al. 1997, Genes and Development. 11:996-1007). These mice express a CD44 -antisense transgene under the control of the keratin-5 promoter which targets expression of the antisense transgene to the basal cell compartment of stratified squamous epithelia.

Derivation and Characterization of Primary Mouse Keratinocytes.

Primary Mouse keratinocyte cultures were established from cells isolated from the epidermis of 1 to 3-day-old mice. Mouse skins were incubated overnight at 4° C. in 0.25% try spin. The epidermis was separated from the dermis, minced and stirred to produce a single cell suspension before seeding onto collagen-coated tissue cultures wells (Nalge Nunc International, Naperville, Ill.). Cell cultures were incubated at 34° C. in 8% CO₂ for 3 to 10 days in serum-free keratinocyte basal medium without calcium, supplemented with KGM Single Quotes, original formula (Clonetics, Inc., San Diego, Calif.). CD44 expression on keratinocytes from each animal was assessed by immunofluorescence microscopy using mAb KM81 (Schrager, H. M. 1998, J. Clin. Invest. 101:1708-1716).

Bacterial adherence assays.

For bacterial adherence assays, keratinocytes were seeded at 10⁵ cells per well in collagen-coated tissue culture wells (Nalge Nunc International, Naperville, Ill.), incubated for 3 days at 34° C. in 8% CO₂, washed, and overlaid with medium containing 10⁶ CFU of GAS per well. After incubation for 1 hour at 34° C. in 8% CO₂ the keratinocyte monolayers were washed twice to remove nonadherent bacteria, then the number of cell-associated bacteria was determined by quantitative culture after releasing the keratinocytes with try spin and lysing them in sterile water, experiments testing the effects of exogenous hyaluronic acid on GAS adherence were performed using the mouse keratinocyte cell line, PAM 2.12 (Song, I. S. 2000, Exp. Dermatol. 9:42-52). Cells were seeded in collagen-coated tissue culture wells at 10⁵ cells per well, cultured for 3 days at 37° C. in 5% CO₂, then inoculated with 10⁶ CFU of GAS per well in medium containing exogenous hyaluronic acid (from rooster comb, Sigma Chemicals, St. Louis, Mo.), no inhibitor, or a control polysaccharide, alginic acid, a high-molecular-weight polymer of mannuronic acid and guluronic acid (Protonal Durvillea Alginate, Pronova. Biopolymer, Inc., Portsmouth, N.H.). Cells were incubated for 45 minutes at 37° C. in 5% CO₂, then processed for enumeration of adherent bacteria as described above. In experiments to test whether exogenous hyaluronic acid could promote dissociation of GAS attached to keratinocytes, GAS were allowed to attach to keratinocytes in the absence of inhibitor; after 45 minutes, the medium was removed and replaced with fresh medium containing hyaluronic acid, no inhibitor, or alginic acid. After 45 minutes of incubation, the number of adherent bacteria was enumerated as described above.

Pharyngeal Colonization Studies in vivo.

For pharyngeal colonization experiments, 4 to 6 week-old female mice were anesthetized by inhalation of methoxyflurane, then inoculated intranasally with approximately 5×10⁶ CFU of GAS in 20 μl phosphate-buffered saline, pH 7.4. Throat swabs were collected from anesthetized mice daily and were plated on Todd-Hewitt-blood agar containing streptomycin 500 μg/ml to inhibit growth of normal flora. For some experiments, the bacterial inoculum was suspended in phosphate-buffered saline containing 50 μg of mAb, either KM81, a rat anti-mouse CD44 mAb (Miyake, K. 1990, J. Exp. Med. 171:477-488), or 4H1, an isotype-matched control monoclonal antibody directed to Pseudomonas aeruginosa lipopolysaccharide (donated by Michael Preston, Brigham and Women's Hospital, Boston, Mass.). Both antibodies were purified by protein G affinity chromatography (GammaBind Plus Sepharose, Amersham Pharmacia Biotech, Piscataway, N.J.) prior to use. Examination by inmmunofluorescence microscopy of GAS cells incubated with mAb revealed no binding of either mAb to the organisms.

Immunohistochemistry.

Paraffin-embedded tissue sections through the mouse pharynx were deparaffinized as described previously (Schrager, H. M. et al. 1998, J. Clin. Invest. 101: 1708-1716). Sections were incubated with 0.1% hydrogen peroxide to quench endogenous peroxidase activity, then incubated for 1 hour with 1.5% normal rabbit serum, then with mAb KM81, 15 μg/ml for 30 minutes at room temperature. Slides were washed with phosphate-buffered saline, then incubated with biotin-conjugated rabbit anti-rat IgG, followed by horseradish peroxidase-streptavidin and the peroxidase substrate, diaminobenzidine tetrahydrochlorlde (Santa Cruz Biotechnology, Inc., Santa Cruz, Calif.). Slides were examined by light microscopy and photographed at 400×magnification under standard conditions.

Statistical analysis.

Differences in attachment of GAS to keratinocytes were evaluated Using the Mann Whitney U test (Instat version 1. 12, GraphPad Software, Inc. San Diego, Cailf.). Repeated measures logistic regression was used to test for the effect of anti-CD44 mAb or erogenous hyaluronic acid treatment on the proportion of mice with positive throat cultures over three days of observation (Zeger, S. L. et al. 1986, Biometrics. 42:121-130).

### Example 1

### GAS Attachment to Murine Keratinocytes in vitro is Mediated by Binding of the GAS Capsular Polysaccharide to CD44

To define the importance of GAS interaction with CD44 in pharyngeal colonization in vivo and to determine whether a murine model would be suitable for this investigation; experiments were preformed to establish that GAS attachment to primary cultured murine keratinocytes, like that to human keratinocytes, was mediated by binding of the GAS capsular polysaccharide to CD44.

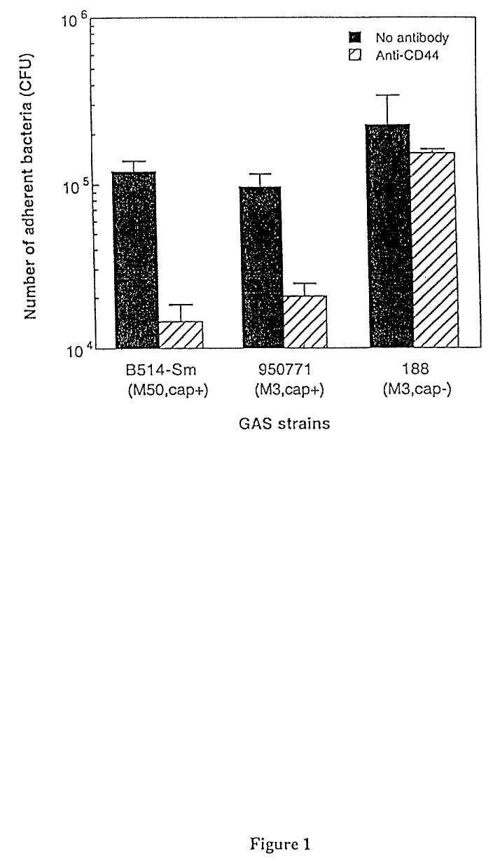

To examine the role of CD44 in GAS binding to murine keratinocytes, primary cultures were established of cells isolated from neonatal C57BL/6 mouse skin. Two GAS strains were studied: B514-Sm, an M-type 50 strain that has been shown to efficiently colonize the upper airway of mice after intranasal inoculation, and 950771, an M-type 3 isolate originally cultured from a patient with necrotizing fascutis and typical of strains that cause human pharyngitis and invasive infection. Monoclonal antibody (mAb) KM81 directed to the hyaluronic acid binding site of mouse CD44 (Zheng, Z., et al. 1995, J. cell Biol. 130:485-495) inhibited by 75% binding of wild-type (i.e., encapsulated) GAS strains.B514-Sm and 950771 to mouse keratinocytes (P<0.005 for both GAS strains compared to binding in the absence of mAb, FIG. 1). Mab KM81 had no significant effect on binding of strain 188, an isogenic acapsular mutant of strain 950771 derived from the wild-type GAS strain by inactivation of the hyaluronate synthase gene, hasA. The overall adherence of the capsule-deficient strain was equal to or greater than that of the encapsulated wild-type strain, but was mediated by binding interactions independent of CD44 . These results are consistent with previous findings that the hyaluronic acid capsule masks alternative adhesins on the bacterial surface and prevents M-protein mediated attachment of GAS to human epithelial cells (Schrager, H. M. et al. 1998, J. Clin. Invest. 101: 1708-1716, Courtney, H. S. et al. 1997, FEMS Microbiol. Let. 151:65-70).

### Example Two

### Encapsulated GAS Attach Poorly to CD44 -Deficient Keratinocytes from Transgenic Mice

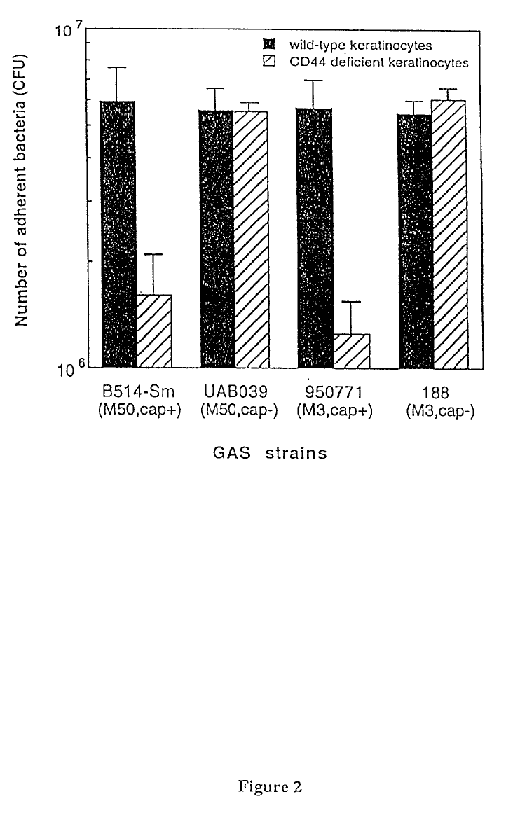

CD44 -deficient transgenic mice were utilized to determine whether the selective loss of CD44 expression by keratinocytes affected GAS binding to keratinocytes in vitro and GAS colonization of the pharynx in vivo. K5-CD44 mice express a CD44 -antisense transgene under the control of the keratin-5 promoter which targets expression of the antisense transgene to the basal cell compartment of stratified squamous epithelia (Kaya, G. 1997, Genes and Development. 11:996-1007). High level expression of the transgene results in complete loss of CD44 expression in all layers of the epidermis. Transgenic animals were screened for high-level expression of the antisense transgene by immunofluorescence microscopy of primary cultured keratinocytes using mAb KM81. The attachment of GAS to primary keratinocytes from wild-type C57BL/6 mice was compared to GAS attachment to K5-CD44 keratinocytes from antisense transgenic animals deficient in CD44 expression. In assays of GAS attachment in vitro, binding of wild-type GAS strains B514-Sm and 950771 to CD44 -deficient keratinocytes was reduced by 73% and 78%, respectively, compared to binding to normal mouse keratinocytes (P<0.01 for binding of both GAS strains to wild-type versus CD44 -deficient keratinocytes, FIG. 2). In contrast, binding of each of two isogenic GAS mutant strains lacking hyaluronic acid capsules to CD44 -deficient keratinocytes was no different than that to normal keratinocytes. These results provide evidence that GAS attachment to mouse keratinocytes in vitro is mediated by binding of the GAS hyaluronic acid capsule to CD44 .

### Example 3

### GAS Colonization of the Airway is Impaired in CD44 -Deficient Transgenic Mice in vivo

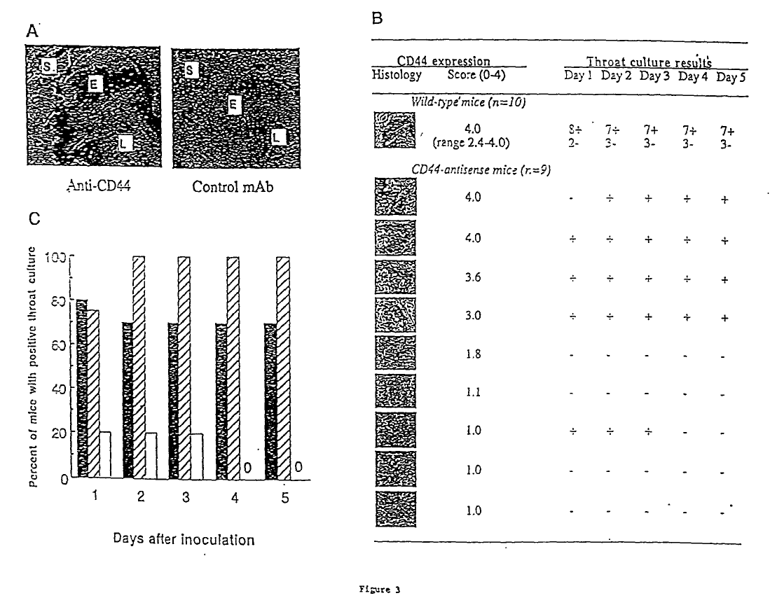

In vivo challenge experiments were performed to determine whether the observed in vitro interaction between the GAS capsule and CD44 reflected a role for CD44 as a receptor for GAS colonization of the pharyngeal epithelium in vivo. Groups of wild-type or K5-CD44 transgenic mice were inoculated intranasally with GAS strain B514-Sm. Throat cultures were obtained daily for 5 days after challenge to assess pharyngeal colonization. The degree of suppression of CD44 expression may be partial or complete in an individual animal depending on the extent of expression of the K5-CD44 antisense transgene. Therefore, the mice were euthanized at the end of the experiment and histologic sections of the pharynx were examined by microscopy after immunohistochemical staining to determine the degree of CD44 expression in the pharyngeal epithelium in individual animals. Four of 9 evaluable transgenic mice exhibited epithelial expression of CD44 similar to the levels in wild-type mice (histologic scores for CD44 expression of 3.0 to 4.0, compared to 2.4 to 4.0 for control mice). In 5 transgenic animals, epithelial CD44 expression was markedly reduced or undetectable (histologic scores of 1.0 to 1.8). Among the wild-type mice, throat cultures were positive for GAS in 8 to 10 animals 1 day after inoculation and in 7 of 10 animals with wild-type levels of CD44 expression: throat cultures were positive in 3 or 4 of 4 mice on each of 5 days after challenge. By contrast, among the 5 transgenic mice with low or absent epithelial CD44 expression, only a single animal had a positive throat culture on days 1, 2, and 3, and all 5 animals had negative cultures on days 4 and 5 (FIG. 3). Therefore, reduced CD44 expression on the pharyngeal epithelium was associated with rapid clearance of the GAS inoculum from the upper airway and failure of the bacteria to colonize the pharynx.

### Example 4

### Blocking GAS Binding to CD44 Prevents GAS Pharyngeal Colonization in Wildtype Mice

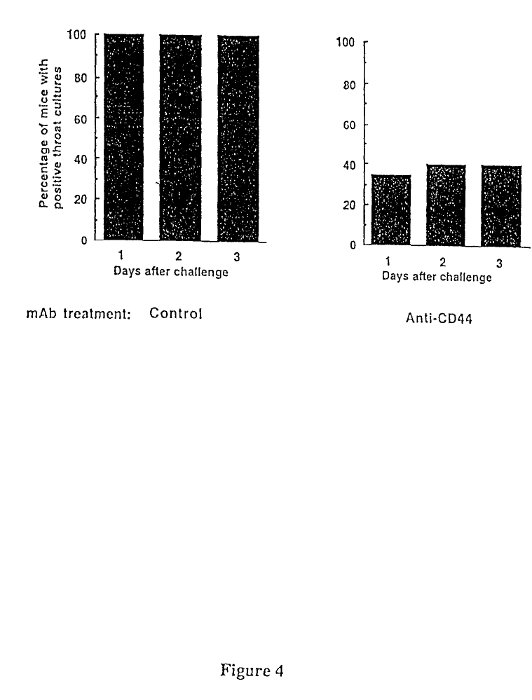

The experiments with transgenic mice described above indicated that pharyngeal expression of CD44 resulted in enhanced colonization by GAS. As an alternative means to characterize the role of CD44 as a GAS receptor, the effect of disrupting the interaction between the GAS capsule and CD44 was investigated in wild-type mice. For these studies, wild-type mice were inoculated intranasally with GAS strain B514-Sm mixed with either mAb to CD44 or an irrelevant control mAb directed to the lipopolysaccharide of Pseudomonas aeruginosa. Intranasal administration of anti-CD44 mAb KM81 together with the GAS challenge reduced colonization by >60% on each of 3 days after inoculation compared to coadministration of the control antibody with the bacterial inoculum (P<0.0001, FIG. 4). Thus, antibody specific for the hyaluronate-binding domain of the CD44 receptor blocked not only GAS attachment to epithelial cells in vitro, but also pharyngeal colonization by GAS in vivo.

### Example 5

### Exogenous Hyaluronic Acid Blocks GAS Binding to Murine Keratinocytes

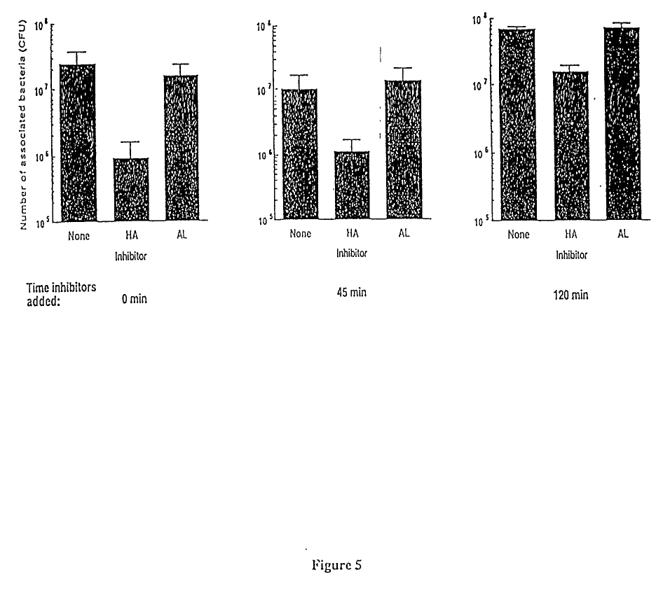

To investigate the specificity of the interaction between the GAS capsule and CD44 on epithelial cells, the ability of exogenous hyaluronic acid to compete with GAS for binding to murine keratinocytes was examined. Addition of exogenous hyaluronic acid to concentration 1 mg/ml/ml, inhibited GAS attachment to the murine keratinocyte cell line PAM2.12 by >95% (P<0.03, FIG. 5). A concentration of 1 /ml alginic acid, another high molecular weight acidic polysaccharide, had no effect, supporting the hypothesis that adherence depends on a specific interaction between the GAS hyaluronic acid capsule and CD44 . Addition of the exogenous hyaluronic acid up to 45 minutes after adding GAS to the keratinocytes still resulted in >90% reduction in bacterial attachment (P<0.03); if addition of hyaluronic acid was delayed for 2 hours, GAS attachment was reduced by 70-80% (P<0.03). These results suggested that the capsule-CD44 binding interaction may be important during the early phase of bacterial attachment, while alternative ligand-receptor interactions may supervene during the later phase.

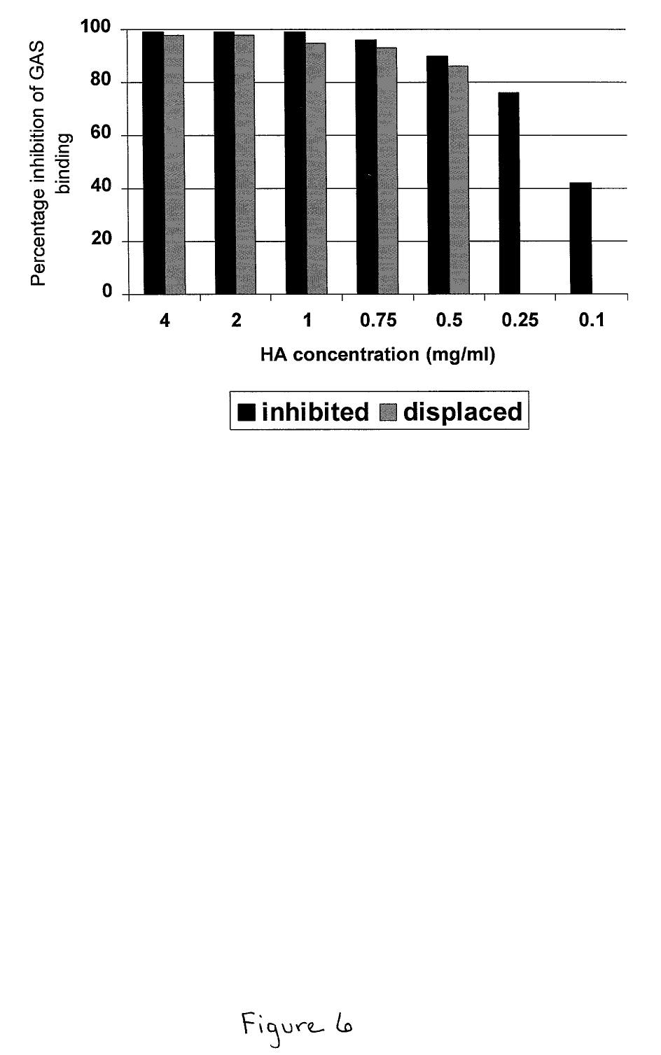

In addition to the 1 mg/ml concentration of hyaluronic acid, additional trials were performed as indicated above, with a range of concentrations of hyaluronic acid added to the cells. The concentrations tested included 4 mg/ml, 2 mg/ml, 1 mg/ml, 0.75 mg/ml, 0.5 mg/ml, 0.25 mg/ml and 0.1 mg/ml. The hyaluronic acid concentrations from 4 mg/ml to 0.25 mg/ml blocked GAS binding by greater than 70%. Hyaluronic acid concentrations from 4 mg/ml to 0.5 mg/ml blocked GAS binding by greater than 80% (FIG. 6).

### Example 6

### Exogenous Hyaluronic Acid Blocks GAS Binding to Human Keratinocytes

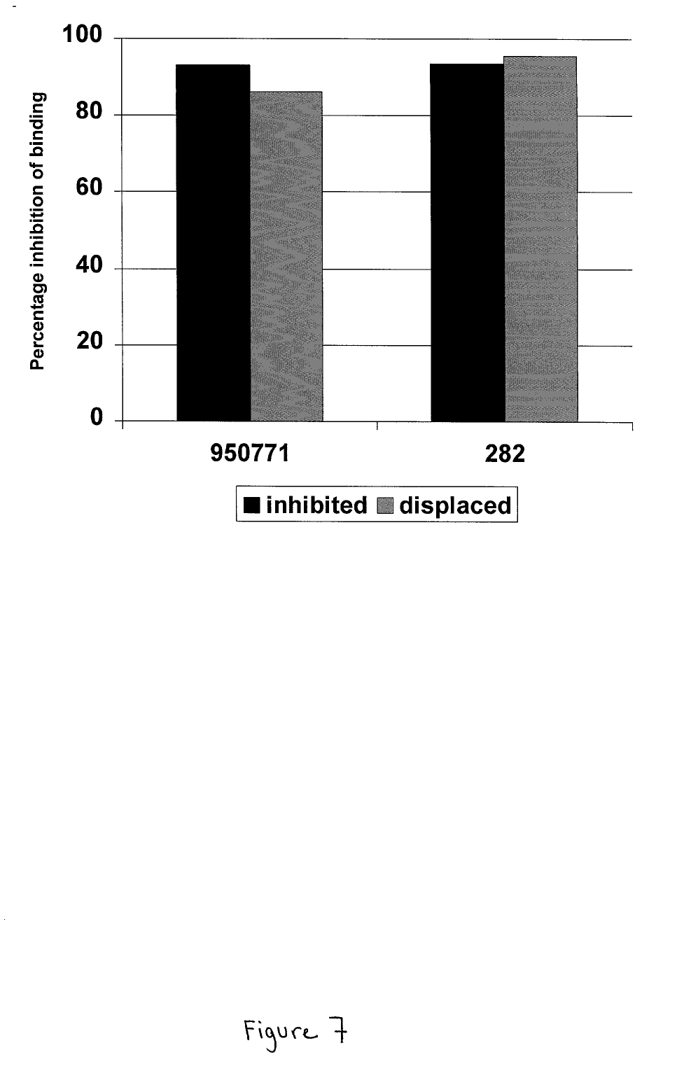

Utilizing the methods described above for preparing murine keratinocyte monolayers, monolayers of human keratinocytes were prepared. The human cell monolayers were inoculated as described above with GAS 950771 or with GAS 282 and 1 mg/ml hyaluronic acid was applied as described above, and the inhibition of binding and displacement of bound bacteria was assessed. Hyaluronic acid was found to block GAS binding to human cells and this effect was found not to be strain specific. (FIG. 7) Hyaluronic acid displaced bound GAS after 45 minutes. The results indicate that hyaluronic acid functioned similarly on human and mouse cells.

### Example 7

### Pretreatment of Mice with Exogenous Hyaluronic Acid Reduces GAS Colonization of the Pharynx

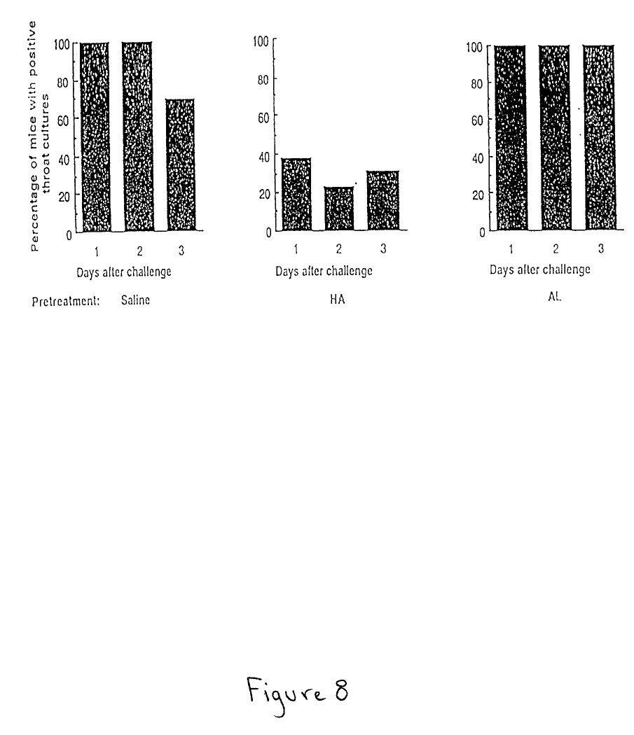

The experiments described above indicate that exogenous hyaluronic acid could compete for GAS binding to epithelial cells in vitro. Whether the same phenomenon might operate in vivo in the mouse model of pharyngeal colonization was examined. Mice were pretreated by intranasal administration of hyaluronic acid (20 μl of a 1 mg/ml solution) 5 hours prior to intranasal inoculation with GAS. Colonization of hyaluronic acid pretreated animals was reduced by approximately 60 to 80% compared to control animals pretreated with phosphate-buffered saline or animals pretreated with alginic acid (P<0.0001, FIG. 8). That hyaluronic acid pretreatment can reduce colonization upon bacterial challenge further supports the hypothesis that interaction with CD44 mediates GAS attachment to the pharyngeal mucosa. Furthermore, it suggests the possibility that topically administered hyaluronic acid or an analog that binds to CD44 might be an effective means of preventing GAS pharyngeal infection during a period of intensive exposure such as an outbreak or epidemic situation.

### Example 8

### Effect of Hyaluronic Acid Added Exogenously in Preventing Translocation of GAS Through Epithelial Barriers

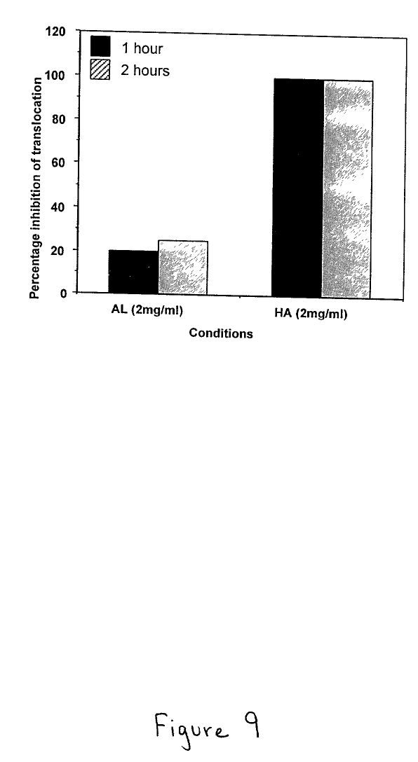

Human keratinocytes were cultured on 3.0 μm filters to allow polarization and establishment of functional intercellular tight junctions. Cells were infected as described previously with wild-type GAS, strain 950771. To assess the ability of exogenously added HA to inhibit the translocation of GAS through the keratinocyte monolayer, cells were pre-treated for 30 minutes with either 2 mg/ml of hyaluronic acid (HA) or another acidic polysaccharide, alginate (AL), at the same concentration. Aliquots of media from the lower chamber of infected keratinocyte monolayers were cultured on blood agar plates at 1 and 2 hours after inoculation with bacteria to assess the number of translocating bacteria. The data are shown in FIG. 9. Pre-treatment with HA resulted in 100% blocking of translocating GAS at both 1 (solid bars) and 2 hours (hatched bars) after inoculation as compared to untreated monolayers. In contrast only 20% and 25% of the wild-type bacteria were blocked by AL at 1 and 2 hours, respectively.

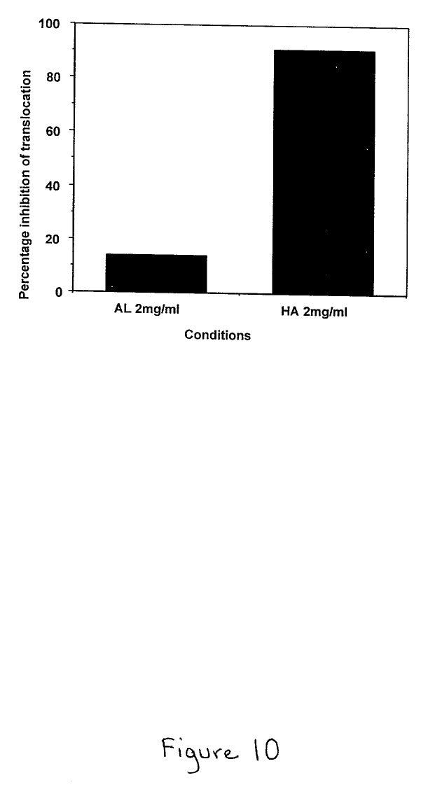

To examine GAS translocation in a model system similar to normal human skin, we utilized pseudo-organ cultures of human skin equivalent. This material is made by constructing a “dermis” that consists of human fibroblasts cultured in a collagen matrix; human foreskin keratinocytes are seeded on top of the dermal equivalent and cultured under conditions that promote differentiation into a stratified squamous epithelium with a stratum comeum. The histology of the human skin equivalent is very similar to that of normal human skin except for the absence of non-keratinocyte elements such as blood vessels and hematopoietic cells, nerves, and hair follicles. Since intact human skin is highly resistant to GAS infection, we simulated minor trauma to the superficial epidermis by abrading the surface of the skin equivalent with a cytology brush to introduce breaks in the stratum corneum. GAS were inoculated onto the surface of abraded skin equivalent that had either been pre-treated with nothing, HA, or AL at 2 mg/ml for 1 hour and translocation through the tissue was monitored by culture of GAS from the basal surface of the skin equivalent at intervals. As depicted in FIG. 10, HA inhibited translocation GAS by 91% (solid bars). In comparison the AL control prevented only 14% of bacteria from translocating through the skin equivalent during the 4 hour infection period.

### Example 9

### Use of Hyaluronic Acid to Inhibit Binding to Human Epithelial Cells by Multiple Bacterial Species

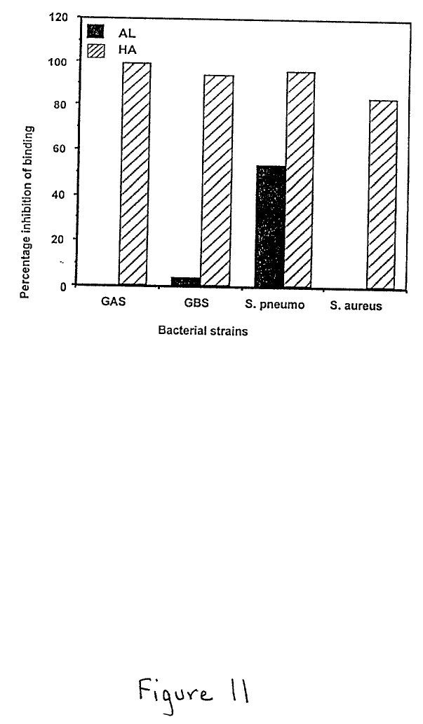

Keratinocyte monolayers were prepared as descibed above in Methods section and pre-incubated with 1 mg/ml alginate (AL, solid bars) or 1 mg/ml hyaluronic acid (HA, hatched bars) prior to surface inoculation (as described above in Methods) with either Group A Streptococcus (GAS, strain 950771), Group B Streptococcus (GBS, strain 515), Streptococcus pneumoniae (S. pneumo, type 6), or Staphylococcus aureus (S. aureus, type 8) at a multiplicity of infection of 10 bacteria per keratinocyte for 1 hour. FIG. 11 indicates that HA pre-treatment inhibited by more than 80% binding of each of the bacterial species tested. These data suggest that topical application of HA at mucosal (eg, throat or nose) or skin sites may prevent infections dues to a variety of pathogenic bacteria.

### Examples 10-12

### METHODS

### Bacterial Strains and Polarized Keratinocyte Monolayers

GAS strains were 950771, an M-type 3 strain originally isolated from a child with necrotizing fasciitis, and 188, an isogenic acapsular mutant derived from 950771 (Ashbaugh, C. D., et al., (1998) J. Clin. Invest. 102, 550-560). Bacteria were cultured in Todd Hewitt broth (Difco, Sparks, Md.) to early exponential phase. For preparation of polarized keratinocyte monolayers, human squamous cell carcinoma cell line, SCC13, derived from cheek epidermis (Cline, P. R. & Rice, R. H. (1983) Cancer Res. 43, 3203-3207) was cultured to confluence in serum-free keratinocyte medium (Gibco BRL, Rockville, Md.) supplemented with 50 μg/ml bovine pituitary extract, 0.1 ng/ml epidermal growth factor and 0.3 mM calcium chloride (c-SFM). Certain experiments were performed also using cell monolayers prepared from OKP7 primary human keratinocytes (Crowe et al. (1991) Differentiation 48, 199-208). Keratinocytes were seeded at 5×10⁵ cells per well onto polycarbonate Transwell membrane supports (12-well plates, 3.0 μm pore size; Costar, Corning, N.Y.) and cultured for 6-10 days at 37° C. with 5% CO₂ in c-SFM, with daily medium changes. Once confluent, the integrity of the monolayer was assessed routinely by measuring permeability to sodium fluorescein (Tchao, R. (1988) Alternative Methods in Toxicology (eds. Goldberg, A. M.) 271-283 (Ann Liebert, N.Y.) and, in some experiments, by measuring transepithelial electrical resistance (McNamara, B. P., et al. (2001) J Clin Invest 107, 621-9).

Antibody Labeling and Confocal Microscopy.

Keratinocyte monolayers were fixed with 4% paraformaldehyde in phosphate buffered saline (PBS), washed, permeabilized with 0.1% Triton X-100 in PBS, then incubated with 0.5% bovine serum albumin in PBS to block non-specific binding sites prior to antibody staining. Primary antibodies included rabbit IgG antibody to GAS group A carbohydrate (ImmuCell, Portland, Me.) at 5 μg/ml, rat monoclonal anti-mouse CD44 (clone IM7.8. 1) (Schrager, H. M., et al., (1998) J. Clin. Invest. 101, 1708-1716) at 15 μg/ml, goat IgG anti-ezrin at 2 μg/ml (Santa Cruz Biotechnology, Santa Cruz, Calif.), goat IgG anti-Rac1 at 2 μg/ml (Santa Cruz Biotechnology), mouse anti-phosphotyrosine at 2 μg/ml (PY-Plus, Zymed Laboratories, Inc., San Francisco, Calif.), rabbit anti-ZO-1 at 2 μg/ml (Zymed), goat anti-E-cadherin at 2 μg/ml (Santa Cruz Biotechnology), and mouse anti-myc monoclonal antibody 9E10 (Santa Cruz Biotechnology). Secondary antibodies included, as appropriate, Texas red- or Alexa 660-conjugated anti-rabbit IgG (Molecular Probes, Eugene, Oreg.), Alexa 568-conjugated anti-rabbit, anti-goat, or anti-rat IgG (Molecular Probes), and FITC-conjugated anti-goat or anti-mouse IgG (Sigma), each diluted 1:250 in 0.5% BSA/PBS. Cellular actin was labeled with 165 nm phalloidin conjugated to Oregon green 514 or to Alexa 488 (Molecular Probes).

Confocal microscopy images were collected using a BioRad scanning confocal microscope equipped with a krypton-argon laser. Images were processed with BioRad Confocal Assistant and Adobe Photoshop 5.5 (Adobe Systems, San Jose, Calif.).

Transfection of SCC13 Keratinocytes with Rac-Constructs.

Eukaryotic expression plasmids pRK5-myc-N17 Rac1 (myc-tagged, dominant negative Rac1) and pRK5-myc-L61 Rac1 (myc-tagged, constitutively active Rac1) were provided by Dr. Alan Hall (University College, London, UK). Polarized SCC13 keratinocyte monolayers were transiently transfected using 5 μg of either expression construct and 30 μl SuperFect transfection reagent (Qiagen, Valencia, Calif.). Twenty-four hours later, the monolayers were inoculated with GAS as described below.

GAS Translocation Through Polarized Keratinocyte Monolayers.

Keratinocyte monolayers on Transwells were inoculated with GAS at a multiplicity of infection of 10 per keratinocyte. Monolayers were incubated at 37° C. with 5% CO₂. The keratinocyte medium in the upper chamber was replaced two hours after adding GAS, and the Transwell inserts containing the keratinocytes were moved to new wells containing fresh medium every 2 hours to prevent overgrowth of translocated bacteria. Translocation was assessed by quantitative cultures of medium from the lower chamber at 2 hour intervals.

Apligraf® living skin equivalent (a gift from Organogenesis, Inc., Canton, Mass.) was used as a model for human skin (Sabolinski, M. L., et al. (1996) Biomaterials 17, 311-20). Samples of skin equivalent were cut into rectangular sections, each approximately 2.5×1.2 cm. Each section was placed on a 12 mm Transwell (Costar, 3.0 μm pore size) and the Transwell was placed on Todd Hewitt agar containing 5% sheep blood. The apical surface of the skin equivalent was lightly brushed with a sterile cytology brush to abrade the stratum corneum, exposing the underlying epidermal keratinocytes. GAS were resuspended at 10⁶ colony-forming units/10 μl in SFM and inoculated onto the surface of the tissue. After inoculation, tissue samples were incubated at 37° C. with 5% CO₂ and no supplemental humidity. Transwells containing the inoculated tissue samples were transferred to fresh blood agar every 2 hrs. The blood agar plates were then incubated overnight at 37° C. for enumeration of colony-forming units representing the number of organisms emerging from the basal surface of the tissue.

### Example 10

### Keratinocyte Monolayer Confocal Microscopy

To investigate the ability of GAS to affect host epithelial cell biology, the interaction of GAS with human keratinocytes, the predominant cell type in skin and the pharyngeal epithelium was examined. Keratinocytes were grown to confluence on porous membrane supports under conditions that induced both the physiologic apical-basal polarization of the epithelial cells and the formation of intercellular junctions (Rheinwald, J. G. (1979) Int Rev Cytol Suppl 10, 25-33; Rheinwald, J. G. (1980) Methods Cell Biol 229-5). These confluent, differentiated cell cultures are referred to as “monolayers” herein, although microscopy showed partial overlap of adjacent cells so that the thickness of the layer in an individual well varied from 1 to 3 cells. Monolayers were inoculated by placement of a suspension of GAS on the apical surface of the cells. Transmission electron microscopy images demonstrated membrane projections from keratinocytes exposed to wild-type GAS strain 950771, but not from cells exposed to an isogenic acapsular mutant, strain 188. Similar membrane ruffles or lamellipodia have been observed in response to localized application of hyaluronic acid to the surface of tumor cells as a consequence of CD44-mediated cell signaling (Oliferenko, S., et al., (2000) [published erratum appears in J Cell Biol Apr. 3, 2000 ;149(1):following 236]. J Cell Biol 148, 1159-64). In confocal microscopy images of monolayers exposed to wild-type GAS, actin filament projections were observed in close association with the bacteria. Triple label experiments in which CD44 was visualized with specific antibody demonstrated that CD44 was distributed along intercellular junctions and at the tips of the actin filament projections, immediately adjacent to bound GAS. These striking microfilament projections were not observed in keratinocytes pretreated with anti-CD44 monoclonal antibody IM7.8.1 prior to inoculation with wild-type GAS, nor in untreated keratinocytes exposed to acapsular GAS, and cell-bound acapsular bacteria were not co-localized with CD44.

### Example 11

### HA Binding in Tumor Cells

In tumor cells, transduction of the extracellular event of hyaluronic acid binding to CD44 to the intracellular movement of actin filaments occurs through activation of Rac1, a member of the Rho family of small GTPases. (Oliferenko, S., et al., (2000) [published erratum appears in J Cell Biol Apr. 3, 2000 ;149(1):following 236]. J Cell Biol 148, 1159-64; Bourguignon, L. Y. et al., (2000) J Biol Chem 275, 1829-38). To determine whether Rac1 was involved in CD44 -mediated cell signaling induced by GAS, keratinocytes were transfected with N17 Rac1 (dominant negative Rac1). Binding of wild-type GAS to N17 Rac1-transfected cells failed to induce formation of lamellipodia, although typical lamellipodia were induced by interaction of GAS with adjacent untransfected cells in the same keratinocyte monolayer. Transfection of keratinocytes with constitutively active L61 Rac1 resulted in lamellipodia formation in uninoculated keratinocytes. Inoculation of these cells with wild-type GAS increased the amount of lamellipodia, and wild-type GAS were observed in close association with the lamellipodia. By contrast, binding of acapsular GAS to L61 Rac1-transfected keratinocytes was not preferentially localized to lamellipodia, but rather occurred equally over regions of the cell devoid of membrane projections. These results demonstrate the role of active Rac1 in the cytoskeletal rearrangements induced by wild-type GAS.

### Example 12

### Co-Localization

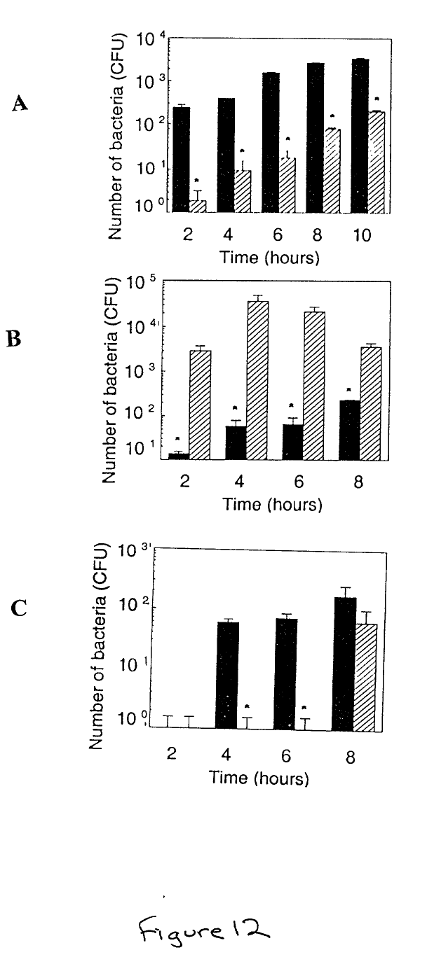

CD44 -mediated cell signaling to the cytoskeleton in other systems also involves ezrin, a protein of the ERM (ezrin-radixin-moesin) family, which is thought to physically link the cytoplasmic domain of CD44 to the actin cytoskeleton upon activation (Tsukita, S. et al., (1994) J Cell Biol 126, 391-401;. Legg, J. W. & Isacke, C. M. (1998) Curr Biol 8, 705-8; Hall, A. (1998) Science 279, 509-14). In keratinocytes exposed to wild-type GAS, confocal microscopy demonstrated co-localization of Rac1 and ezrin precisely at the point of attachment of GAS to the keratinocyte surface (FIG. 12a-b). By contrast, binding of acapsular GAS to keratinocytes was not associated with co-localization of Rac1 or ezrin with the bound bacteria. These results suggest that binding of the GAS hyaluronic acid capsule to keratinocyte CD44 triggers the local activation of Rac1 and association of the actin-linker protein ezrin with the cytoplasmic domain of CD44 , physically coupling the keratinocyte cell membrane, via CD44 and ezrin, to the actin cytoskeleton. Thus, Rac1-mediated actin polymerization could result in local movement of the cell membrane to form lamellipodia and to open intercellular junctions with neighboring cells (see below herein). Ligand binding to CD44 is expected to activate cellular protein tyrosine kinases as part of the downstream intracellular signaling cascade. (Bourguignon, L. Y. W. et al., (2001) J. Biol. Chem. 276, 7327-7336; Taher, T. E., et al. (1996) J Biol Chem 271, 2863-7). Consistent with this model, confocal microscopy revealed co-localization of antibody to tyrosine-phosphorylated protein(s) with CD44 at the site of attachment of wild-type, but not acapsular, GAS to the keratinocyte surface. Incubation of keratinocytes with the protein tyrosine kinase inhibitor, genistein, was also found to inhibit formation of lamellipodia in response to GAS, evidence that tyrosine phosphorylation of an as yet unidentified protein may serve to couple CD44 -signaling to Rac1 activation.

Scanning electron microscopy and confocal microscopy images of keratinocyte monolayers exposed to GAS showed not only evidence of localized membrane projections, but also lifting of the margin of the keratinocyte in the vicinity of associated GAS, with curling back of the cell membrane from its contacts with the subtratum and with the neighboring cell. Similar morphologic changes have been observed in epithelial cells subjected to localized application of hyaluronic acid or to microinjection with constitutively active Rac1. (Oliferenko, S. et al., (2000) [published erratum appears in J Cell Biol Apr. 3, 2000 ;149(1):following 236]. J Cell Biol 148, 1159-64; Hall, A.(1998) Science 279, 509-14). In several instances, GAS organisms were seen, deep to the surface of the monolayer, within the intercellular breach. Such disruptions of intercellular junctions were not observed in keratinocytes exposed to acapsular GAS. Investigation was made to determine whether GAS-induced disruption of intercellular junctions was associated with changes in distribution of the tight junction-associated protein ZO-1 or of E-cadherin, a member of the cadherin family of transmembrane proteins that mediate cell-cell adhesion through homophilic interaction of their extracellular domains on adjacent cells. Immunofluorescent staining demonstrated striking loss of both ZO-1 and E-cadherin from intercellular junctions after exposure of keratinocytes to wild-type, but not acapsular, GAS. This redistribution was prevented by pretreatment of the keratinocytes with genistein, implicating tyrosine kinase(s) in the process. These findings provide further evidence that interaction of the GAS hyaluronic acid capsule with CD44 results in Rac1-dependent disruption of intercellular junctions. Loss of integrity of intercellular junctions is expected to reduce the barrier function of the monolayer, as reflected by its ability to prevent the passage of small molecules such as sodium fluorescein. Infection of monolayers with the encapsulated wildtype strain resulted in an increase in permeability to sodium fluorescein from <3% to >30% and a corresponding 33% decrease in transepithelial electrical resistance, after 6 hours of exposure, whereas infection with the acapsular mutant strain resulted in no significant change in the monolayer barrier function.

That GAS binding to CD44 induced destabilization and opening of intercellular junctions suggested that these defects in epithelial integrity might facilitate translocation of the bacteria through the epithelium. As a model of the movement of GAS from the pharyngeal mucosa or skin surface into underlying tissue, GAS translocation was studied in vitro through keratinocyte monolayers. Two hours after inoculation onto the surface of a confluent keratinocyte monolayer, wild-type GAS was detected in the culture medium below the membrane support, indicating migration of the organisms through the monolayer. At each of several intervals after inoculation, approximately 20 to 100 times as many GAS were recovered below monolayers inoculated with wild-type GAS compared to those inoculated with the acapsular mutant strain.(Figure 12a). A comparable difference in efficiency of translocation between wild-type and acapsular GAS was observed in separate experiments using monolayers of primary human keratinocytes. Compared to wild-type GAS strain 950771, the efficiency of translocation was similar for wild-type GAS strains 87-282, a highly encapsulated M-type 18 strain, and for DLS003, a poorly encapsulated M-type 3 strain. Translocation of wild-type GAS was reduced by 78% at 1 hour in experiments in which the keratinocyte monolayers were pretreated with monoclonal antibody IM7.8.1 to block interaction of the GAS capsule with CD44 . Similarly, in the presence of 250 μM genistein, the earliest detectable translocation of wild-type GAS was delayed from 30 min to 1 hr, and the maximum number of translocated GAS at 4 hours was reduced by approximately 80%.

Transmission electron microscopy demonstrated wild-type GAS predominantly at extracellular sites. Organisms were seen on the apical surface of the cells in association with lamellipodia and in intercellular spaces. By contrast, organisms of the acapsular mutant strain that were visualized on the surface of keratinocytes appeared to be tightly apposed to the keratinocyte cell membrane and not associated with lamellipodia; in addition, many acapsular bacteria were observed within keratinocytes in membrane-bound vacuoles. These results were confirmed by quantitative cultures of organisms recovered from cell lysates of infected keratinocytes. At 2 hours after inoculation of the keratinocyte monolayer, approximately 200-fold more intracellular GAS were recovered from monolayers infected with the acapsular mutant than from monolayers infected with the wild-type strain. Similar results were obtained in studies of monolayers infected for 4, 6, or 8 hours. (FIG. 12b). Thus, the efficiency of translocation through the monolayer was inversely related to entry of the bacteria into the keratinocytes. Trypan blue staining did not reveal gross cytopathic changes in the keratinocytes during the period of the translocation assays. Furthermore, in assays measuring release of LDH as a marker of cell injury, greater amounts were released from keratinocytes exposed to acapsular compared to wild-type GAS. Therefore, the increased translocation of wild-type compared to acapsular GAS was not attributable to a greater cytotoxic effect by the wild-type bacteria.

To examine GAS translocation in a model system similar to normal human skin, pseudoorgan cultures of human skin equivalent (Parenteau, N. L. et al. (1992) Cytotechnology 9, 163-71; Sabolinski, M. L. et al., (1996) Biomaterials 17, 311-20) were utilized. Histology of the human skin equivalent is very similar to that of normal human skin, a differentiated, stratified squamous epithelium that consists of multiple cell layers. Because intact human skin is highly resistant to GAS infection, minor trauma to the superficial epidermis was simulated by abrading the surface of the skin equivalent with a cytology brush to introduce breaks in the stratum comeum. GAS were inoculated onto the surface of abraded skin equivalent and translocation through the tissue was monitored by culture of GAS from the basal surface of the skin equivalent at intervals. Wild-type GAS were detected from the basal surface of the skin equivalent 4 hours after surface inoculation, whereas acapsular GAS were not detected until 8 hours after inoculation (FIG. 12c). Examination of histologic sections by confocal microscopy demonstrated wild-type GAS in intercellular spaces throughout the epidermis and dermis, whereas acapsular GAS were visualized primarily in the superficial layers of the epidermis, often within keratinocytes. Thus, capsule-deficient organisms fail to translocate efficiently, but rather remained trapped within keratinocytes in the superficial epidermis. These findings extend similar results obtained using keratinocyte monolayers to a system with important features of normal human skin.

EQUIVALENTS

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference.

What is claimed is:

1. A method for treating a subject to reduce the likelihood of streptococcal infection comprising
administering orally to a subject in need of such treatment an agent that binds to a hyaluronic acid-binding region of a CD44 protein in an amount effective to interfere with adhesion of streptococcal bacteria to CD44 protein in the subject and inhibit streptococcal colonization of the pharynx, wherein either one or both of the following conditions applies: the treatment is free of Echinacea or the agent is administered in a dose greater than 0.2 mg.

2. The method of claim 1, wherein the effective amount of the agent administered to the subject in need of such treatment statistically reduces the likelihood of infection.

3. The method of claim 1, wherein the agent is administered to a subject suspected of exposure to Group A streptococcus or Group C streptococcus.

4. The method of claim 3, wherein the subject is known to have been exposed to streptococcal bacteria.

5. The method of claim 3, wherein the subject is likely to be exposed to streptococcal bacteria.

6. The method of claim 1, wherein the subject is a human.

7. The method of claim 1, wherein the subject is in need of such treatment to reduce the likelihood of streptococcal pharyngitis.

8. The method of claim 1, wherein the CD44 protein in the subject in need of such treatment is located on the pharynx.

9. The method of claim 1, wherein the agent is hyaluronic acid or an analog of hyaluronic acid.

10. The method of claim 9, wherein the agent is a peptide.

11. The method of claim 10, wherein the peptide is an antibody.

12. The method of claim 1, wherein the dose is a single oral administration of hyaluronic acid.

13. The method of claim 1, wherein the dose is multiple oral administrations of hyaluronic acid.

14. The method of claim 1, wherein a dose of at least 0.2 mg is administered in under 2 hours.

15. The method of claim 1, wherein a dose of at least 0.2 mg is administered in under 1 hour.

16. The method of claim 1, wherein a dose of at least 0.2 mg is administered in under 30 minutes.

17. The method of claim 1, wherein a dose of at least 0.2 mg is administered in under 15 minutes.

18. The method of claims 14, 15, 16, or 17, wherein the dose is at least 0.25 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 10.0 mg hyaluronic acid.

19. The method of claim 14, wherein the dose is administered in under 1.5 hours, 1.0 hours, 45 minutes, 30 minutes, 15 minutes, 10 minutes, 5 minutes, 4 minutes, 3 minutes, 2 minutes, 1 minutes, 30 seconds, 15 seconds, 10 seconds, 5 seconds, or 1 second.

20. The method of claims 2, 3, 4, 5, 6, 7, 8, 9, 10, 11, 12, 13, 14, 15, 16, 17, or 19, wherein the treatment is free of Echinacea.

21. The method of claim 18, wherein the treatment is free of Echinacea.

22. The method of claim 1, wherein the treatment is free of Echinacea.

23. A method for treating a subject to reduce the likelihood of streptococcal infection comprising
administering orally to a subject in need of such treatment an agent that binds to a hyaluronic acid-binding region of a CD44 protein in an amount effective to displace streptococcal bacteria to CD44 protein in the subject and inhibit streptococcal colonization of the pharynx, wherein either one or both of the following conditions applies: the treatment is free of Echinacea or the agent is administered in a dose greater than 0.2 mg.

24. The method of claim 23, wherein the effective amount of the agent administered to the subject in need of such treatment statistically reduces the likelihood of infection.

25. The method of claim 23, wherein the agent is administered to a subject suspected of exposure to Group A streptococcus or Group C streptococcus.

26. The method of claim 25, wherein the subject has not been determined to have streptococcal infection.

27. The method of claim 25, wherein the subject has been determined to have streptococcal infection.

28. The method of claim 23, wherein the subject is a human.

29. The method of claim 23, wherein the subject is in need of such treatment to reduce the likelihood of streptococcal pharyngitis.

30. The method of claim 23, wherein the CD44 protein in the subject in need of such treatment is located on the pharynx.

31. The method of claim 23, wherein the agent is hyaluronic acid or an analog of hyaluronic acid.

32. The method of claim 31, wherein the agent is a peptide.

33. The method of claim 32, wherein the peptide is an antibody.

34. The method of claim 23, wherein the dose is a single oral administration of hyaluronic acid.

35. The method of claim 23, wherein the dose is multiple oral administrations of hyaluronic acid.

36. The method of claim 23, wherein a dose of at least 0.2 mg is administered in under 2 hours.

37. The method of claim 23, wherein a dose of at least 0.2 mg is administered in under 1 hour.

38. The method of claim 23, wherein a dose of at least 0.2 mg is administered in under 30 minutes.

39. The method of claim 23, wherein a dose of at least 0.2 mg is administered in under 15 minutes.

40. The method of claims 36, 37, 38, or 39, wherein the dose is at least 0.25 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 10.0 mg hyaluronic acid.

41. The method of claim 36, wherein the dose is administered in under 1.5 hours, 1.0 hours, 45 minutes, 30 minutes, 15 minutes, 10 minutes, 5 minutes, 4 minutes, 3 minutes, 2 minutes, 1 minutes, 30 seconds, 15 seconds, 10 seconds, 5 seconds, or 1 second.

42. The method of claims 24,25,26,27,28,29,30, 31,32, 33,34,35,36,37,38,39, or 41, wherein the treatment is free of Echinacea.

43. The method of claim 40, wherein the treatment is free of Echinacea.

44. The method of claim 23, wherein the treatment is free of Echinacea.

45. A method for treating a subject to reduce the likelihood of streptococcal or staphylococcal infection comprising
administering orally to a subject in need of such treatment an agent that binds to a hyaluronic acid-binding region of a CD44 protein of a mucosal membrane in an amount effective to interfere with adhesion of streptococcal or staphylococcal bacteria to the mucosal membrane in the subject, wherein either one or both of the following conditions applies: the treatment is free of Echinacea or the agent is administered in a dose greater than 0.2 mg.

46. The method of claim 45, wherein the hyaluronic acid interferes with adhesion of streptococcal bacteria to CD44 protein of a mucosal membrane in the subject.

47. The method of claim 45, wherein the effective amount of the agent administered to the subject in need of such treatment statistically reduces the likelihood of infection.

48. The method of claim 45, wherein the agent is administered to a subject suspected of exposure to Group A streptococcus, Group C streptococcus, Group B streptococcus, Streptococcus pneumoniae, or Staphylococcus aureus.

49. The method of claim 48, wherein the subject is known to have been exposed to streptococcal or staphylococcal bacteria.

50. The method of claim 48, wherein the subject is likely to be exposed to streptococcal or staphylococcal bacteria.

51. The method of claim 45, wherein the subject is a human.

52. The method of claim 45, wherein the subject is in need of such treatment to reduce the likelihood of streptococcal or staphylococcal pharyngitis.

53. The method of claim 45, wherein the CD44 protein of the mucosal membrane in the subject in need of such treatment is located on the pharynx.

54. The method of claim 45, wherein the agent is hyaluronic acid or an analog of hyaluronic acid.

55. The method of claim 54, wherein the agent is a peptide.

56. The method of claim 55, wherein the peptide is an antibody.

57. The method of claim 45, wherein the dose is a single oral administration of hyaluronic acid.

58. The method of claim 45, wherein the dose is multiple oral administrations of hyaluronic acid.

59. The method of claim 45, wherein a dose of at least 0.2 mg is administered in under 2 hours.

60. The method of claim 45, wherein a dose of at least 0.2 mg is administered in under 1 hour.

61. The method of claim 45, wherein a dose of at least 0.2 mg is administered in under 30 minutes.

62. The method of claim 45, wherein a dose of at least 0.2 mg is administered in under 15 minutes.

63. The method of claims 59, 60, 61, or 62, wherein the dose is at least 0.25 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 10.0 mg hyaluronic acid.

64. The method of claim 59, wherein the dose is administered in under 1.5 hours, 1.0 hours, 45 minutes, 30 minutes, 15 minutes, 10 minutes, 5 minutes, 4 minutes, 3 minutes, 2 minutes, 1 minutes, 30 seconds, 15 seconds, 10 seconds, 5 seconds, or 1 second.

65. The method of claims 46, 47, 48, 49, 50, 51, 52, 53, 54, 55, 56, 57, 58, 59, 60, 61, 62, or 64, wherein the treatment is free of Echinacea.

66. The method of claim 63, wherein the treatment is free of Echinacea.

67. The method of claim 45, wherein the treatment is free of Echinacea.

68. A method for treating a subject to reduce the likelihood of streptococcal or staphylococcal infection comprising
administering orally to a subject in need of such treatment an agent that binds to a hyaluronic acid-binding region of a CD44 protein of a mucosal membrane in an amount effective to displace streptococcal or staphylococcal bacteria bound to the mucosal membrane in the subject, wherein either one or both of the following conditions applies: the treatment is free of Echinacea or the agent is administered in a dose greater than 0.2 mg.

69. The method of claim 68, wherein the hyaluronic acid interferes with adhesion of streptococcal bacteria to CD44 protein of a mucosal membrane in the subject.

70. The method of claim 68, wherein the effective amount of the agent administered to the subject in need of such treatment statistically reduces the likelihood of infection.

71. The method of claim 68, wherein the agent is administered to a subject suspected of exposure to Group A streptococcus, Group C streptococcus, Group B streptococcus, Streptococcus pneumoniae, or Staphylococcus aureus.

72. The method of claim 71, wherein the subject has not been determined to have been exposed to streptococcal or staphylococcal infection.

73. The method of claim 71, wherein the subject has been determined to have been exposed to streptococcal or staphylococcal infection.

74. The method of claim 68, wherein the subject is a human.

75. The method of claim 68, wherein the subject is in need of such treatment to reduce the likelihood of streptococcal or staphylococcal pharyngitis.

76. The method of claim 68, wherein the CD44 protein of the mucosal membrane in the subject in need of such treatment is located on the pharynx.

77. The method of claim 68, wherein the agent is hyaluronic acid or an analog of hyaluronic acid.

78. The method of claim 77, wherein the agent is a peptide.

79. The method of claim 78, wherein the peptide is an antibody.

80. The method of claim 68, wherein the dose is a single oral administration of hyaluronic acid.

81. The method of claim 68, wherein the dose is multiple oral administrations of hyaluronic acid.

82. The method of claim 68, wherein a dose of at least 0.2 mg is administered in under 2 hours.

83. The method of claim 68, wherein a dose of at least 0.2 mg is administered in under 1 hour.

84. The method of claim 68, wherein a dose of at least 0.2 mg is administered in under 30 minutes.

85. The method of claim 68, wherein a dose of at least 0.2 mg is administered in under 15 minutes.

86. The method of claims 82, 83, 84, or 85, wherein the dose is at least 0.25 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 110.0 mg hyaluronic acid.

87. The method of claim 82, wherein the dose is administered in under 1.5 hours, 1.0 hours, 45 minutes, 30 minutes, 15 minutes, 10 minutes, 5 minutes, 4 minutes, 3 minutes, 2 minutes, 1 minutes, 30 seconds, 15 seconds, 10 seconds, 5 seconds, or 1 second.

88. The method of claims 69, 70, 71, 72, 73, 74, 75, 76, 77, 78, 79, 80, 81, 82, 83, 84, 85, or 87, wherein the treatment is free of Echinacea.

89. The method of claim 86, wherein the treatment is free of Echinacea.

90. The method of claim 68, wherein the treatment is free of Echinacea.

91. A method for treating a subject to reduce the likelihood of streptococcal or staphylococcal infection comprising
administering nasally to a subject in need of such treatment who does not have a nasal cavity wound, an agent that binds to a hyaluronic acid-binding region of a CD44 protein of a mucosal membrane in an amount effective to interfere with adhesion of streptococcal or staphylococcal bacteria to the mucosal membrane in the subject, wherein either one or both of the following conditions applies: the treatment is free of Echinacea or the agent is administered in a dose greater than 0.2 mg.

92. The method of claim 91, wherein the hyaluronic acid interferes with adhesion of streptococcal bacteria to CD44 protein of a mucosal membrane in the subject.

93. The method of claim 91, wherein the CD44 protein of the mucosal membrane in the subject in need of such treatment is on a nasal mucosal membrane.

94. The method of claim 91, wherein the effective amount of the agent administered to the subject in need of such treatment statistically reduces the likelihood of infection.

95. The method of claim 91, wherein the agent is administered to a subject suspected of exposure to Group A streptococcus, Group C streptococcus, Group B streptococcus, Streptococcus pneumoniae, or Staphylococcus aureus.

96. The method of claim 95, wherein the subject is known to have been exposed to streptococcal or staphylococcal bacteria.

97. The method of claim 95, wherein the subject is likely to be exposed to streptococcal or staphylococcal bacteria.

98. The method of claim 91, wherein the subject is a human.

99. The method of claim 91, wherein the subject is in need of such treatment to reduce the likelihood of streptococcal or staphylococcal pharyngitis.

100. The method of claim 91, wherein the CD44 protein of the mucosal membrane in the subject in need of such treatment is located on the pharynx.

101. The method of claim 91, wherein the agent is hyaluronic acid or an analog of hyaluronic acid.

102. The method of claim 101, wherein the agent is a peptide.

103. The method of claim 102, wherein the peptide is an antibody.

104. The method of claim 91, wherein the dose is a single oral administration of hyaluronic acid.

105. The method of claim 91, wherein the dose is multiple oral administrations of hyaluronic acid.

106. The method of claim 91, wherein a dose of at least 0.2 mg is administered in under 2 hours.

107. The method of claim 91, wherein a dose of at least 0.2 mg is administered in under 1 hour.

108. The method of claim 9 1, wherein a dose of at least 0.2 mg is administered in under 30 minutes.

109. The method of claim 91, wherein a dose of at least 0.2 mg is administered in under 15 minutes.

110. The method of claims 106, 107, 108, or 109, wherein the dose is at least 0.25 mg, 0.30 mg, 0.40 mg, 0.50 mg, 0.60 mg, 0.70 mg, 0.80 mg, 0.90 mg, 5.0 mg, 1.25 mg, 1.55 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 10.0 mg hyaluronic acid.

111. The method of claim 106, wherein the dose is administered in under 1.5 hours, 1.0 hours, 45 minutes, 30 minutes, 15 minutes, 10 minutes, 5 minutes, 4 minutes, 3 minutes, 2 minutes, 1 minutes, 30 seconds, 15 seconds, 10 seconds, 5 seconds, or 1 second.

112. The method of claims 92, 93, 94, 95, 96, 97, 98, 99, 100, 101, 102, 103, 104, 105, 106, 107, 108, 109, or 112, wherein the treatment is free of Echinacea.

113. The method of claim 110, wherein the treatment is free of Echinacea.

114. The method of claim 91, wherein the treatment is free of Echinacea.

115. A method for treating a subject to reduce the likelihood of streptococcal or staphylococcal infection comprising
administering nasally to a subject in need of such treatment who does not have a nasal cavity wound, an agent that binds to a hyaluronic acid-binding region of a CD44 protein of a mucosal membrane in an amount effective to displace streptococcal or staphylococcal bacteria bound to the mucosal membrane in the subject, wherein either one or both of the following conditions applies: the treatment is free of Echinacea or the agent is administered in a dose greater than 0.2 mg.

116. The method of claim 115, wherein the hyaluronic acid interferes with adhesion of streptococcal bacteria to CD44 protein of a mucosal membrane in the subject.

117. The method of claim 115, wherein the CD44 protein of the mucosal membrane in the subject in need of such treatment is on a nasal mucosal membrane.

118. The method of claim 115, wherein the effective amount of the agent administered to the subject in need of such treatment statistically reduces the likelihood of infection.

119. The method of claim 115, wherein the agent is administered to a subject suspected of exposure to Group A streptococcus, Group C streptococcus, Group B streptococcus, Streptococcus pneumoniae, or Staphylococcus aureus.

120. The method of claim 119, wherein the subject has not been determined to have been exposed to streptococcal or staphylococcal infection.

121. The method of claim 119, wherein the subject has been determined to have been exposed to streptococcal or staphylococcal infection.

122. The method of claim 115, wherein the subject is a human.

123. The method of claim 115, wherein the subject is in need of such treatment to reduce the likelihood of streptococcal or staphylococcal pharyngitis.

124. The method of claim 115, wherein the CD44 protein of the mucosal membrane in the subject in need of such treatment is located on the pharynx.

125. The method of claim 115, wherein the agent is hyaluronic acid or an analog of hyaluronic acid.

126. The method of claim 125, wherein the agent is a peptide.

127. The method of claim 126, wherein the peptide is an antibody.

128. The method of claim 115, wherein the dose is a single oral administration of hyaluronic acid.

129. The method of claim 115, wherein the dose is multiple oral administrations of hyaluronic acid.

130. The method of claim 115, wherein a dose of at least 0.2 mg is administered in under 2 hours.

131. The method of claim 115, wherein a dose of at least 0.2 mg is administered in under 1 hour.

132. The method of claim 115, wherein a dose of at least 0.2 mg is administered in under 30 minutes.

133. The method of claim 115, wherein a dose of at least 0.2 mg is administered in under 15 minutes.

134. The method of claims 130, 131, 132, or 133, wherein the dose is at least 0.25 mg, 0.30 mg, 0.40 mg, 0.50 mg, 0.60 mg, 0.70 mg, 0.80 mg, 0.90 mg, 1.0 mg, 1.25 mg, 1.5 mg, 1.75 mg, 2.0 mg, 2.25 mg, 2.5 mg, 2.75 mg, 3.0 mg, 4.0 mg. 5.0 mg, 6.0 mg, 7.0 mg, 8.0 mg, 9.0 mg, or 10.0 hyaluronic acid.

135. The method of claim 130, wherein the dose is administered in under 1.5 hours, 1.0 hours, 45 minutes, 30 minutes, 15 minutes, 10 minutes, 5 minutes, 4 minutes, 3 minutes, 2 minutes, 1 minutes, 30 seconds, 15 seconds, 10 seconds, 5 seconds, or 1 second.

136. The method of claims 116, 117, 118, 119, 120, 121, 122, 123, 124, 125, 126, 127, 128, 129, 130, 131, 132, 133, or 135, wherein the treatment is free of Echinacea.

137. The method of claim 134, wherein the treatment is free of Echinacea.

138. The method of claim 115, wherein the treatment is free of Echinacea.

139. A medicinal product comprising a syrup containing an amount of hyaluronic acid effective to interfere with adhesion of streptococcal bacteria to CD44 protein in a subject and to inhibit streptococcal colonization of a pharynx of the subject.

140. A medicinal product comprising a syrup containing an amount of hyaluronic acid effective to displace streptococcal bacteria bound to CD44 protein in a subject and to inhibit streptococcal colonization of a pharynx of the subject.

141. A medicinal product comprising a syrup containing an amount of hyaluronic acid effective to interfere with adhesion of streptococcal or staphylococcal bacteria to a mucosal membrane in a subject.

142. The medicinal product of claim 141, wherein the mucosal membrane in the subject is a pharynx of the subject.

143. A medicinal product comprising a syrup containing an amount of hyaluronic acid effective to displace streptococcal or staphylococcal bacteria bound to a mucosal membrane in a subject.

144. The medicinal product of claim 143, wherein the mucosal membrane in the subject is a pharynx of the subject.

145. A medicinal product comprising a frozen solution containing an amount of hyaluronic acid effective to interfere with adhesion of streptococcal bacteria to CD44 protein in a subject and to inhibit streptococcal colonization of a pharynx of the subject.

146. A medicinal product comprising a frozen solution containing an amount of hyaluronic acid effective to displace streptococcal bacteria bound to CD44 protein in a subject and to inhibit streptococcal colonization of a pharynx in the subject.

147. A medicinal product comprising a frozen solution containing an amount of hyaluronic acid effective to interfere with adhesion of streptococcal or staphylococcal bacteria to a mucosal membrane in a subject.

148. The medicinal product of claim 147, wherein the mucosal membrane in the subject is a pharynx of the subject.

149. A medicinal product comprising a frozen solution containing an amount of hyaluronic acid effective to displace streptococcal or staphylococcal bacteria bound to a mucosal membrane in a subject.

150. The medicinal product of claim 149, wherein the mucosal membrane in the subject is a pharynx of the subject.

151. A medicinal product comprising a solid solution containing an amount of hyaluronic acid effective to interfere with adhesion of streptococcal bacteria to CD44 protein in a subject and to inhibit streptococcal colonization of a pharynx of the subject.

152. A medicinal product comprising a solid solution containing an amount of hyaluronic acid effective to displace streptococcal bacteria bound to CD44 protein in a subject and to inhibit streptococcal colonization of a pharynx of the subject.

153. A medicinal product comprising a solid solution containing an amount of hyaluronic acid effective to interfere with adhesion of streptococcal or staphylococcal bacteria to a mucosal membrane in a subject.

154. The medicinal product of claim 153, wherein the mucosal membrane in the subject is a pharynx of the subject.

155. A medicinal product comprising a solid solution containing an amount of hyaluronic acid effective to displace streptococcal or staphylococcal bacteria bound to a mucosal membrane in the subject.

156. The medicinal product of claim 155, wherein the mucosal membrane in the subject is a pharynx of the subject.

157. A medicinal product comprising a semi-solid solution containing an amount of hyaluronic acid effective to interfere with adhesion of streptococcal bacteria to CD44 protein in a subject and to inhibit streptococcal colonization of a pharynx of the subject.

158. A medicinal product comprising a semi-solid solution containing an amount of hyaluronic acid effective to displace streptococcal bacteria bound to CD44 protein in a subject and to inhibit streptococcal colonization of a pharynx of the subject.

159. A medicinal product comprising a semi-solid solution containing an amount of hyaluronic acid effective to interfere with adhesion of streptococcal or staphylococcal bacteria to a mucosal membrane in a subject.

160. The medicinal product of claim 159, wherein the mucosal membrane in the subject is a pharynx of the subject.

161. A medicinal product comprising a semi-solid solution containing an amount of hyaluronic acid effective to displace streptococcal or staphylococcal bacteria bound to a mucosal membrane in a subject.

162. The medicinal product of claim 161, wherein the mucosal membrane in the subject is a pharynx of the subject.
